(19) United States
(12) Patent Application Publication
(10) Pub. No.: US 2023/0406829 A1
(43) Pub. Date: Dec. 21, 2023

(54) Heterocyclic Compound and Preparation Method and Use Thereof

(71) Applicant: SYNTRON (JIANGSU) PHARMACEUTICAL TECHNOLOGY CO., LTD., Yancheng, Jiangsu (CN)

(72) Inventors: Sheng Jiang, Yancheng, Jiangsu (CN); Yibei Xiao, Yancheng, Jiangsu (CN); Tianyu Wang, Yancheng, Jiangsu (CN); Yu Zhang, Yancheng, Jiangsu (CN); Haiping Hao, Yancheng, Jiangsu (CN); Minmin Wang, Yancheng, Jiangsu (CN); Kuojun Zhang, Yancheng, Jiangsu (CN); Yatao Qiu, Yancheng, Jiangsu (CN)

(21) Appl. No.: 18/035,770

(22) PCT Filed: Aug. 27, 2021
(86) PCT No.: PCT/CN2021/115172

§ 371 Date: May 8, 2023

(30) Foreign Application Priority Data
Nov. 6, 2020 (CN) 202011228233.9

(51) Int. Cl. C07D 235/18; C07D 405/04; C07D 471/04; C07D 487/04; C07D 491/048; C07D 277/66; C07D 263/57; C07D 491/056; C07D 403/10; A61P 35/00

(52) U.S. Cl. CPC C07D 235/18; C07D 405/04; C07D 471/04; C07D 487/04; C07D 491/048; C07D 277/66; C07D 263/57; C07D 491/056; C07D 403/10; A61P 35/00

(57) ABSTRACT

Disclosed are a heterocyclic compound for an immune checkpoint inhibitor capable of blocking a VISTA signaling pathway, and a preparation method therefor and a use thereof. The compound is as represented by formula I. The compound has a novel structure, can be formulated for oral administration, overcomes the treatment defects and drug resistance of monoclonal antibody-based immune checkpoint inhibitors, can be used to prepare a small molecule inhibitor easily, and is convenient for industrial production.

### TECHNICAL FIELD

The present invention belongs to a biomedical technology, which specifically relates to heterocyclic compounds capable of immune checkpoint inhibitors blocking the VISTA signaling pathway and their preparation methods and pharmaceutical use.

### BACKGROUND

Malignant tumors are a class of diseases that seriously threaten human health and life. At present, cancer treatment includes surgery, radiotherapy, chemotherapy and targeted therapy. Cancer immunotherapy refers to a therapeutic method to improve the anti-tumor immune effect by stimulating the body's immune system, so as to suppress and kill tumor cells. The research of immunotherapy has a history of nearly one hundred years. With the comprehensive development and cross-penetration of oncology, immunology and molecular biology, immunotherapy has achieved many results, bringing new hope for tumor treatment.

Immune checkpoint inhibitors are currently relatively popular immunotherapy drugs. By upregulating the expression of the immune checkpoint receptor, tumor cells inhibit the T cell activity of immune cells and complete the immune escape of tumor cells. Immune checkpoint inhibitors are to inhibit the immune checkpoint pathway, relieve the inhibition of immune cells, activate the body's immune killing of tumor cells, and achieve the effect of tumor therapy. Currently, the identified immune checkpoints are CTLA-4 (cytotoxic T lymphocyte-associated antigen-4), PD-1 (Programmed cell death 1), TIM 3 and so on (T cell membrane 3) (see Drew M. Pardoll, Nature Review Cancer, 2012, 12, 252).

V-Domain Ig Suppressor of T Cell Activation (VISTA) is a class of immune checkpoints expressed in hematopoietic cell tissues. VISTA was also highly expressed in marrow cells, neurogenic cells, and neutrophils. Unlike other immune checkpoints that induce expression after activation of the immune response, VISTA is stably expressed during the period when the immune cells are in homeostasis. Human VISTA consists of 279 amino acids, and the extracellular domain and PD-L1 are homologous, also known as PD-1 homologous protein (PD-1H). Several studies in VISTA-deficient mice have shown that VISTA-deficient mice are susceptible to autoimmune diseases. Therefore, the inhibition of VISTA signaling pathway can repair the anti-tumor immune activity of the body, and the inhibitor research targeting VISTA signaling pathway has also become a research focus. To date, there are still no small-molecule inhibitors of the VISTA signaling pathway on the market. Therefore, the development of novel VISTA small molecule inhibitors with good anticancer activity is of great significance.

### Invention Content

Purpose: Currently, no small-molecule inhibitors of VISTA pathway are on the market. The present invention provides VISTA small molecule inhibitor compound and its different method and pharmaceutical use.

Technical solution: To achieve the above purpose, the invention discloses a heterocyclic compound as shown in formula I below, with pharmaceutically acceptable salts, racemers, optical isomers or solvent compound:

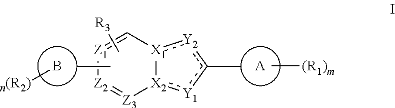

Loops A and B are independently aromatic or aromatic;
X₁, X₂, Z₁, Z₂, Z₃ independently for either C or N, Y₁, Y₂ independently for C, N, S or O;
Each R₁ independently is selected from hydrogen, deuterium, substituted or unsubstituted hydroxyl, substituted or unsubstituted amino, halogen, substituted or unsubstituted alkyl or substituted or unsubstituted alkoxy, amino acids;
Each R₂ independent is selected from hydrogen, deuterium or unsubstituted hydroxyl, substituted or unsubstituted amino, halogen, substituted or unsubstituted alkyl, substituted or unsubstituted alkoxy, or two adjacent R₂ forming substituted or unsubstituted C₄₋₇ carbon ring or heterocycles with two atoms of the B ring;
R₃ independent is selected from hydrogen, deuterium, cyanide, halogen, vinyl, trifluoromethyl, methoxy, or C₁₋₄ alkyl;
m is 1, 2, or 3;
n is 1 or 2.

Further, for each of the R₁, the substituted alkyl or substituted alkoxgroup can be one or more of the following groups: halogen, C₁₋₄ alkyl, hydroxyl groups, and C₁₋₄ alkyxy, cyanide, trifluoromethyl, C₁₋₄ carboxyl, C₁₋₄ ester group or C₁₋₄ amide group; the substituent in the substituted hydroxyl or substituted amino group can be one or more of the following groups: C₁₋₈ alkyl, C₁₋₈ acylamino, C₁₋₈ ester group, C₁₋₈ carboxyl, C₁₋₈ hydroxyl group; wherein the C₁₋₈ alkyl, C₁₋₈ acylamino, C₁₋₈ ester group, C₁₋₈ carboxyl, C₁₋₈ hydroxyl group may optionally be substituted by one or more of the following substituents: hydroxyl, carboxyl, cyanide, amino, cyclic alk, aryl, heterocyclic, alkenyl, alkyne group; when the substituents are multiple, the substituents are the same or different.

Further, for each of the R₂, the substituted alkyl or substituted alkoxy group can be one or more of the following groups: halogen, C₁₋₄ alkyl, hydroxyl groups, and C₁₋₄ alkyxy, cyanide, trifluoromethyl, C₁₋₄ carboxyl, C₁₋₄ ester group or C₁₋₄ amide group; the substituent in the substituted hydroxyl or substituted amino group is one or more of the following groups: C₁₋₈ alkyl, C₁₋₈ acylamino, C₁₋₈ ester group, C₁₋₈ carboxyl, C₁₋₈ hydroxyl group; wherein the C₁₋₈ alkyl, C₁₋₈ acylamino, C₁₋₈ ester group, C₁₋₈ carboxyl, C₁₋₈ hydroxyl group may optionally be substituted by one or more of the following substituents: hydroxyl, carboxyl, cyanide, amino, cycloalkyl, aryl, heterocyclic, alkenyl, alkyne; when the two adjacent R2 and the two atoms in the B ring form a 4-7 substituted carbon ring or substituted heterocyclic, the substituted carbon ring or substituted heterocyclic substituent is one or more of the following groups: Halogens, C₁₋₄ alkyl, hydroxyl groups, and C₁₋₄ alkoxy, cyanide, trifluoromethyl, C₁₋₄ carboxyl, C₁₋₄ ester group or C₁₋₄ amide group; when the substituents are multiple, the substituent is the same or different.

Preferably, the compounds are selected from the following compounds 1-64:

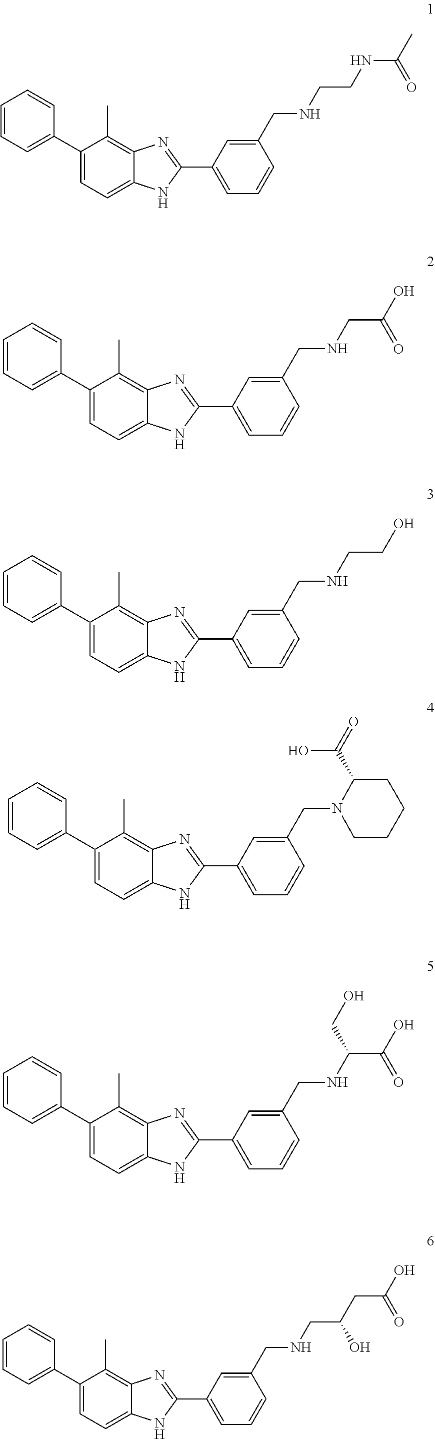 [SMILES: CC(=O)NCCNCc1cccc(-c2nc3c(C)c(-c4ccccc4)ccc3[nH]2)c1 ; Cc1c(-c2ccccc2)ccc2[nH]c(-c3cccc(CNCC(=O)O)c3)nc12 ; Cc1c(-c2ccccc2)ccc2[nH]c(-c3cccc(CNCCO)c3)nc12 ; Cc1c(-c2ccccc2)ccc2[nH]c(-c3cccc(CN4CCCC[C@H]4C(=O)O)c3)nc12 ; Cc1c(-c2ccccc2)ccc2[nH]c(-c3cccc(CN[C@H](CO)C(=O)O)c3)nc12 ; Cc1c(-c2ccccc2)ccc2[nH]c(-c3cccc(CNC[C@@H](O)CC(=O)O)c3)nc12]

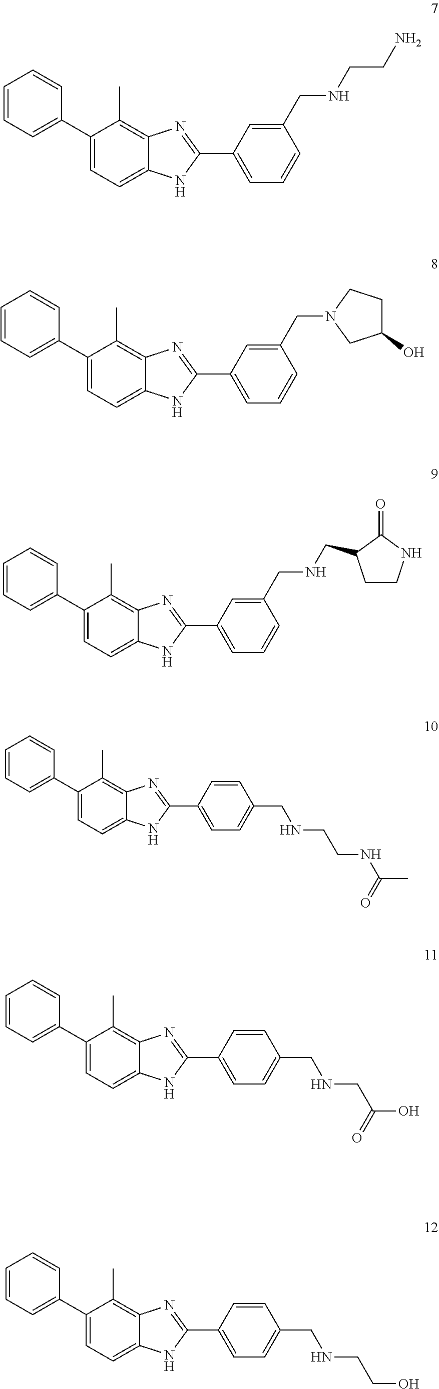

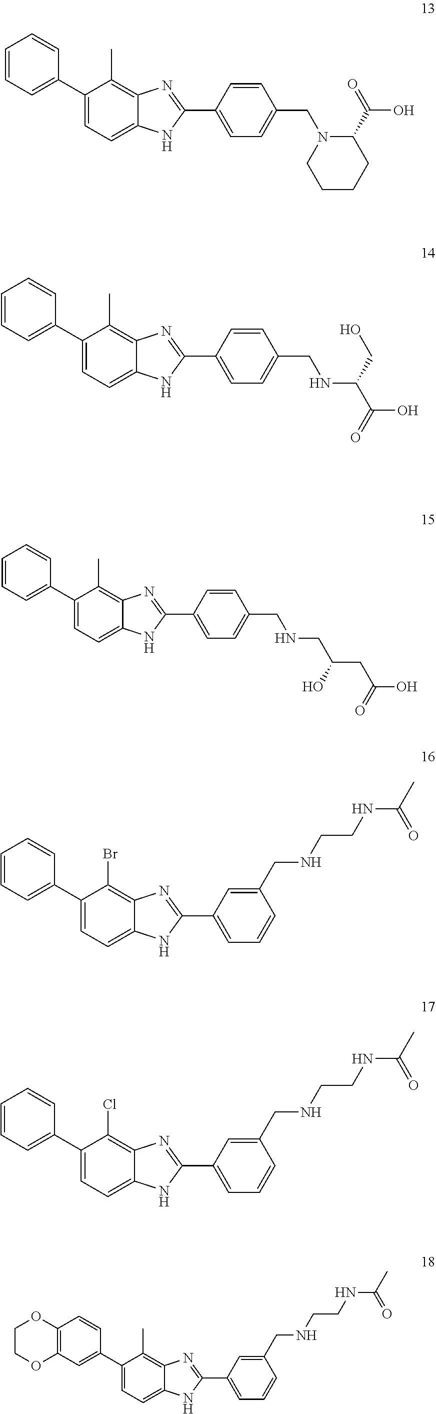 [SMILES: Cc1c(-c2ccccc2)ccc2[nH]c(-c3ccc(CN4CCCC[C@H]4C(=O)O)cc3)nc12 ; Cc1c(-c2ccccc2)ccc2[nH]c(-c3ccc(CN[C@H](CO)C(=O)O)cc3)nc12 ; Cc1c(-c2ccccc2)ccc2[nH]c(-c3ccc(CNC[C@@H](O)CC(=O)O)cc3)nc12 ; CC(=O)NCCNCc1cccc(-c2nc3c(Br)c(-c4ccccc4)ccc3[nH]2)c1 ; CC(=O)NCCNCc1cccc(-c2nc3c(Cl)c(-c4ccccc4)ccc3[nH]2)c1 ; CC(=O)NCCNCc1cccc(-c2nc3c(C)c(-c4ccc5c(c4)OCCO5)ccc3[nH]2)c1]

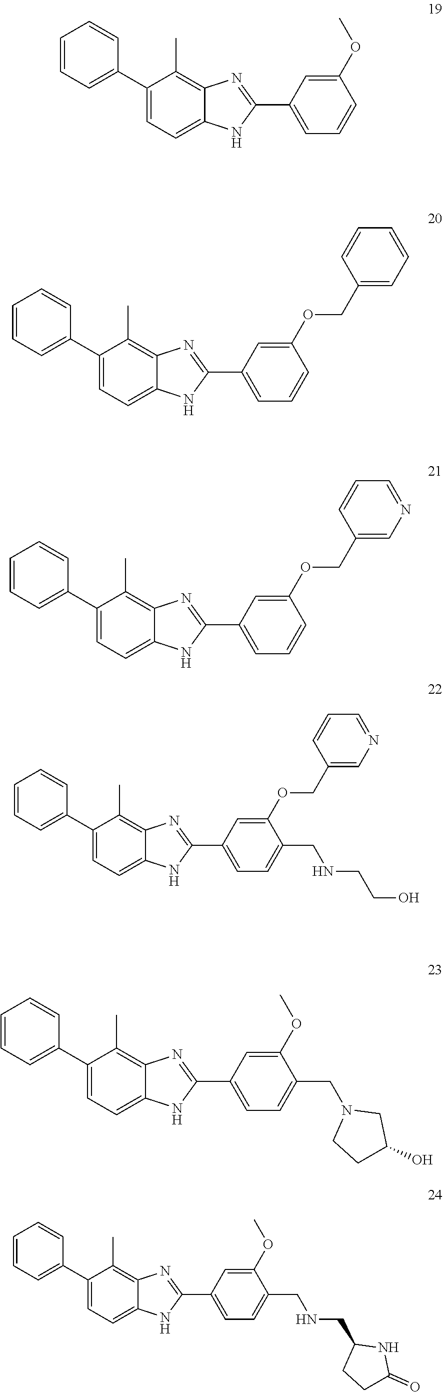 [SMILES: COc1cccc(-c2nc3c(C)c(-c4ccccc4)ccc3[nH]2)c1 ; Cc1c(-c2ccccc2)ccc2[nH]c(-c3cccc(OCc4ccccc4)c3)nc12 ; Cc1c(-c2ccccc2)ccc2[nH]c(-c3cccc(OCc4cccnc4)c3)nc12 ; Cc1c(-c2ccccc2)ccc2[nH]c(-c3ccc(CNCCO)c(OCc4cccnc4)c3)nc12 ; COc1cc(-c2nc3c(C)c(-c4ccccc4)ccc3[nH]2)ccc1CN1CC[C@@H](O)C1 ; COc1cc(-c2nc3c(C)c(-c4ccccc4)ccc3[nH]2)ccc1CNC[C@@H]1CCC(=O)N1]

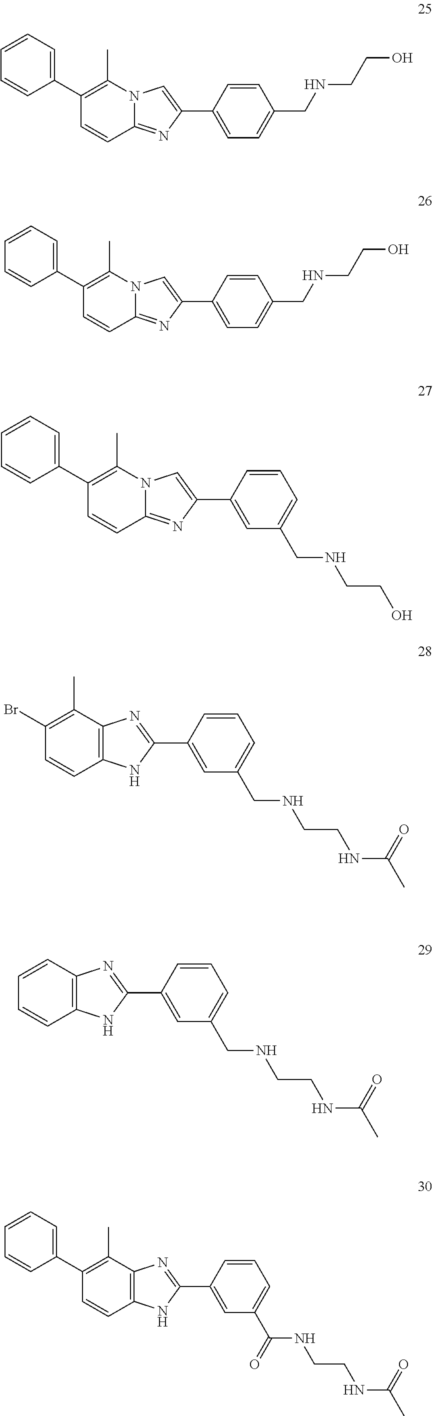 [SMILES: Cc1c(-c2ccccc2)ccc2nc(-c3ccc(CNCCO)cc3)cn12 ; Cc1c(-c2ccccc2)ccc2nc(-c3ccc(CNCCO)cc3)cn12 ; Cc1c(-c2ccccc2)ccc2nc(-c3cccc(CNCCO)c3)cn12 ; CC(=O)NCCNCc1cccc(-c2nc3c(C)c(Br)ccc3[nH]2)c1 ; CC(=O)NCCNCc1cccc(-c2nc3ccccc3[nH]2)c1 ; CC(=O)NCCNC(=O)c1cccc(-c2nc3c(C)c(-c4ccccc4)ccc3[nH]2)c1]

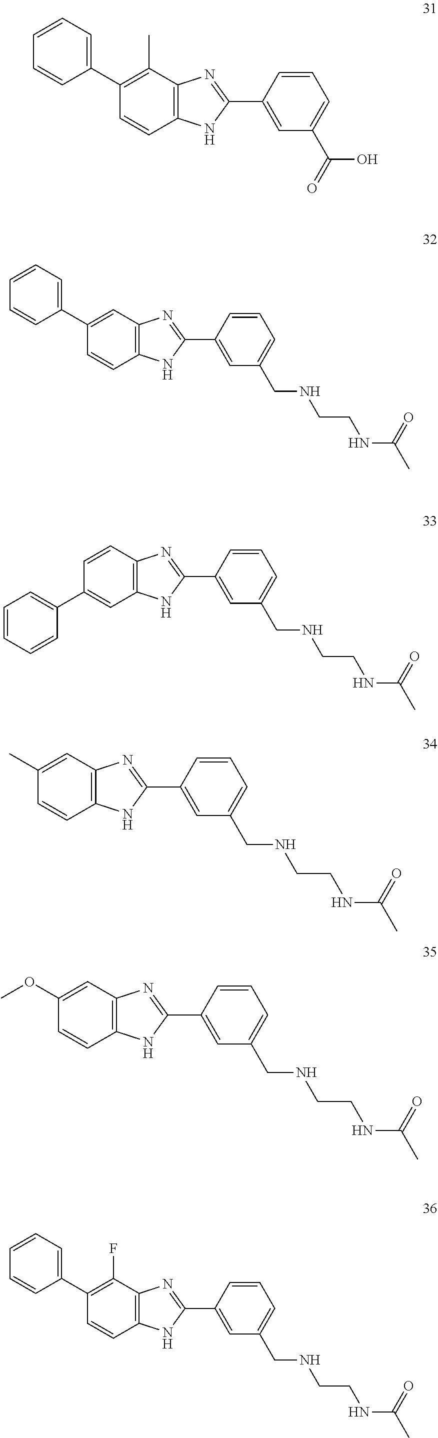 [SMILES: Cc1c(-c2ccccc2)ccc2[nH]c(-c3cccc(C(=O)O)c3)nc12 ; CC(=O)NCCNCc1cccc(-c2nc3cc(-c4ccccc4)ccc3[nH]2)c1 ; CC(=O)NCCNCc1cccc(-c2nc3ccc(-c4ccccc4)cc3[nH]2)c1 ; CC(=O)NCCNCc1cccc(-c2nc3cc(C)ccc3[nH]2)c1 ; COc1ccc2[nH]c(-c3cccc(CNCCNC(C)=O)c3)nc2c1 ; CC(=O)NCCNCc1cccc(-c2nc3c(F)c(-c4ccccc4)ccc3[nH]2)c1]

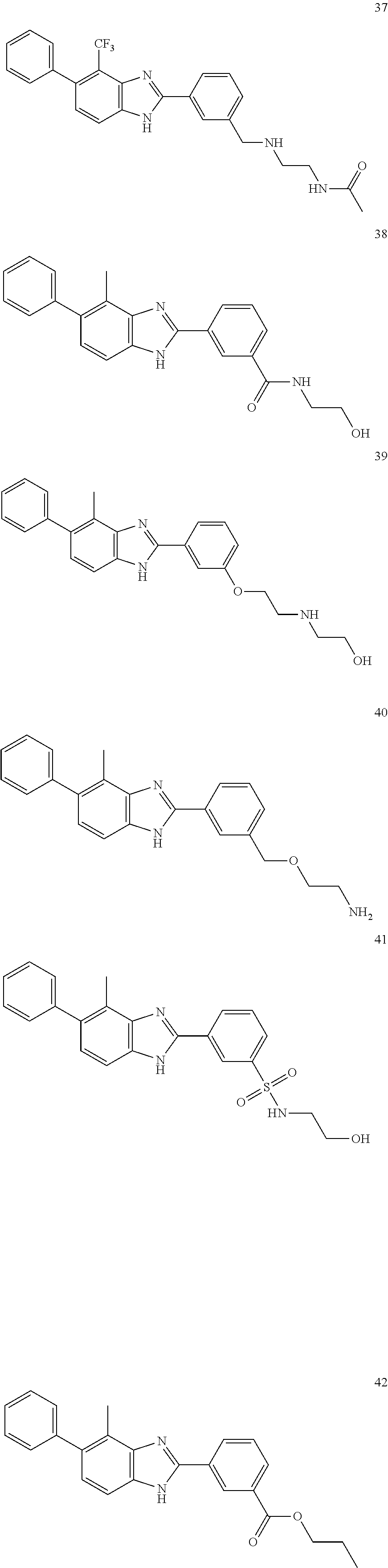 [SMILES: CC(=O)NCCNCc1cccc(-c2nc3c(C(F)(F)F)c(-c4ccccc4)ccc3[nH]2)c1 ; Cc1c(-c2ccccc2)ccc2[nH]c(-c3cccc(C(=O)NCCO)c3)nc12 ; Cc1c(-c2ccccc2)ccc2[nH]c(-c3cccc(OCCNCCO)c3)nc12 ; Cc1c(-c2ccccc2)ccc2[nH]c(-c3cccc(COCCN)c3)nc12 ; Cc1c(-c2ccccc2)ccc2[nH]c(-c3cccc(S(=O)(=O)NCCO)c3)nc12 ; CCCOC(=O)c1cccc(-c2nc3c(C)c(-c4ccccc4)ccc3[nH]2)c1]

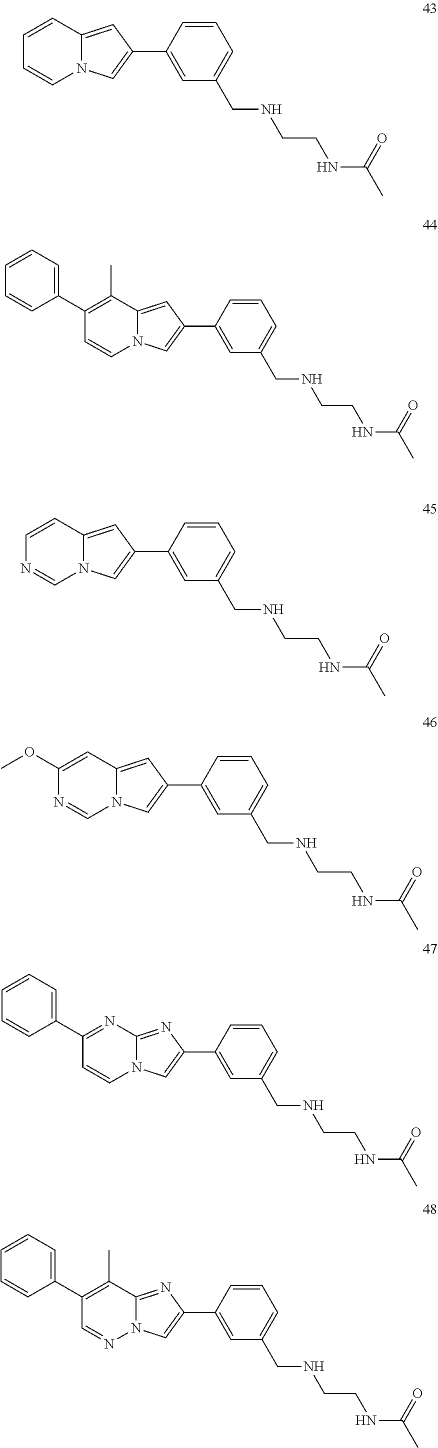 [SMILES: CC(=O)NCCNCc1cccc(-c2cc3ccccn3c2)c1 ; CC(=O)NCCNCc1cccc(-c2cc3c(C)c(-c4ccccc4)ccn3c2)c1 ; CC(=O)NCCNCc1cccc(-c2cc3ccncn3c2)c1 ; COc1cc2cc(-c3cccc(CNCCNC(C)=O)c3)cn2cn1 ; CC(=O)NCCNCc1cccc(-c2cn3ccc(-c4ccccc4)nc3n2)c1 ; CC(=O)NCCNCc1cccc(-c2cn3ncc(-c4ccccc4)c(C)c3n2)c1]

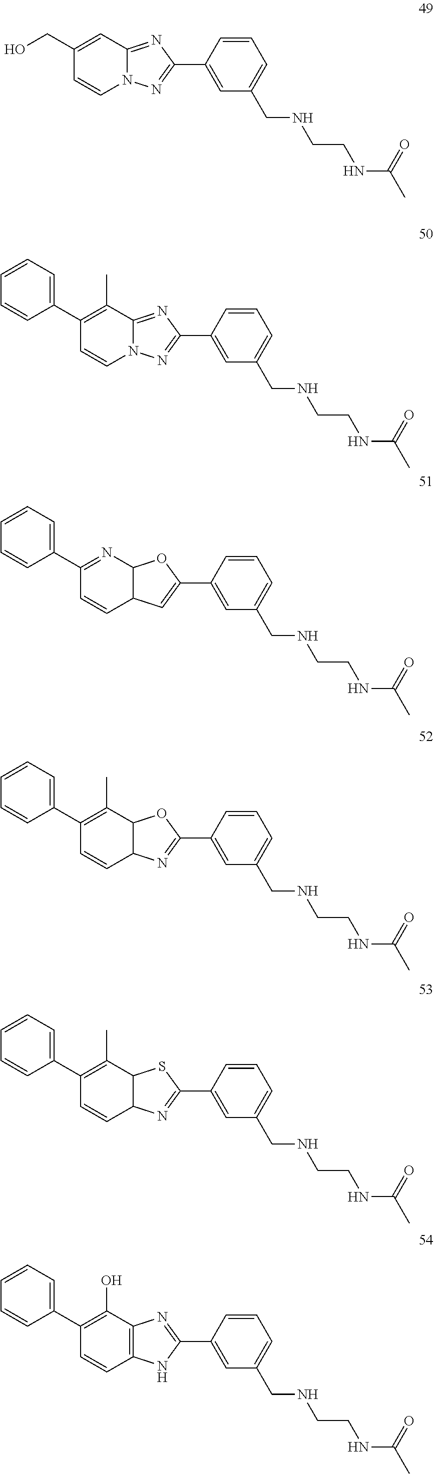 [SMILES: CC(=O)NCCNCc1cccc(-c2nc3cc(CO)ccn3n2)c1 ; CC(=O)NCCNCc1cccc(-c2nc3c(C)c(-c4ccccc4)ccn3n2)c1 ; CC(=O)NCCNCc1cccc(C2=CC3C=CC(c4ccccc4)=NC3O2)c1 ; CC(=O)NCCNCc1cccc(C2=NC3C=CC(c4ccccc4)=C(C)C3O2)c1 ; CC(=O)NCCNCc1cccc(C2=NC3C=CC(c4ccccc4)=C(C)C3S2)c1 ; CC(=O)NCCNCc1cccc(-c2nc3c(O)c(-c4ccccc4)ccc3[nH]2)c1]

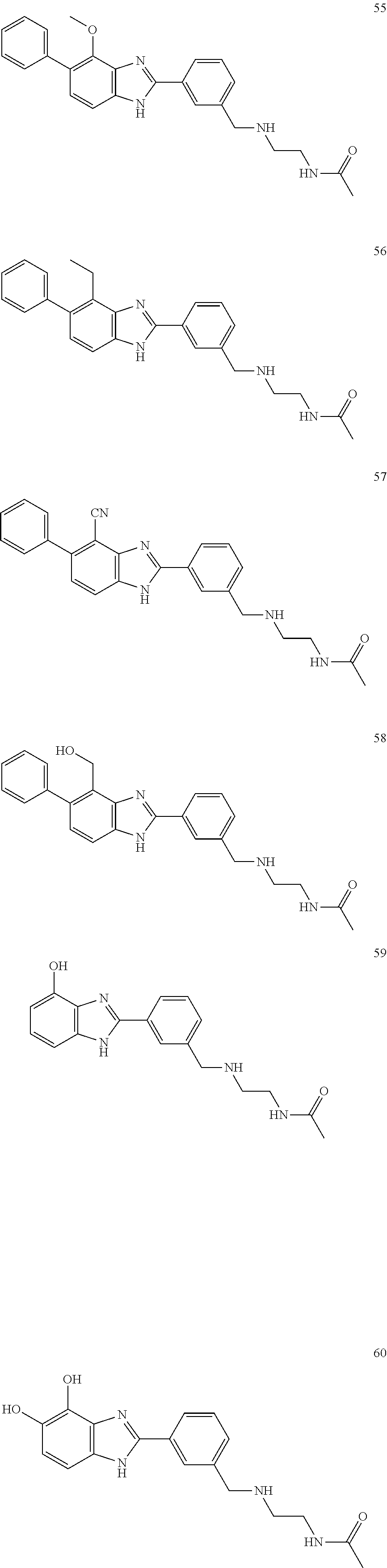 [SMILES: COc1c(-c2ccccc2)ccc2[nH]c(-c3cccc(CNCCNC(C)=O)c3)nc12 ; CCc1c(-c2ccccc2)ccc2[nH]c(-c3cccc(CNCCNC(C)=O)c3)nc12 ; CC(=O)NCCNCc1cccc(-c2nc3c(C#N)c(-c4ccccc4)ccc3[nH]2)c1 ; CC(=O)NCCNCc1cccc(-c2nc3c(CO)c(-c4ccccc4)ccc3[nH]2)c1 ; CC(=O)NCCNCc1cccc(-c2nc3c(O)cccc3[nH]2)c1 ; CC(=O)NCCNCc1cccc(-c2nc3c(O)c(O)ccc3[nH]2)c1]

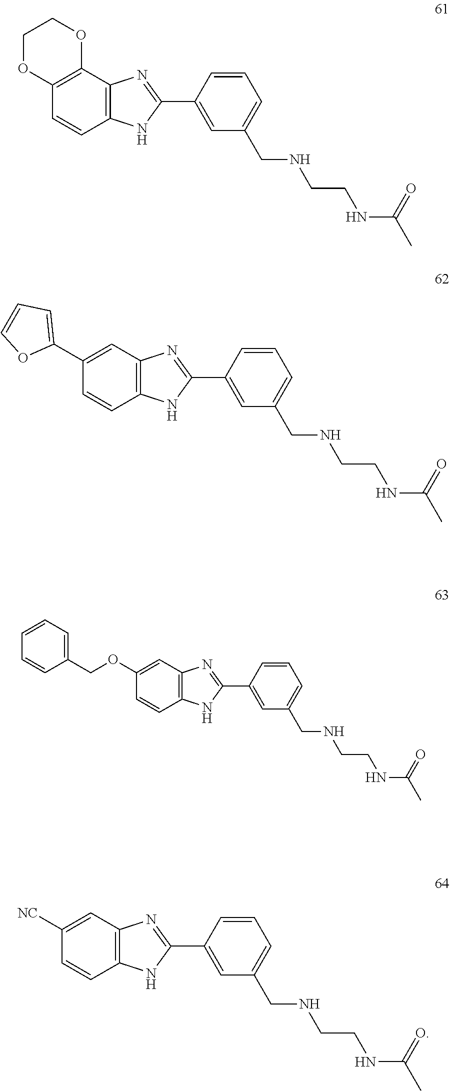 [SMILES: CC(=O)NCCNCc1cccc(-c2nc3c4c(ccc3[nH]2)OCCO4)c1 ; CC(=O)NCCNCc1cccc(-c2nc3cc(-c4ccco4)ccc3[nH]2)c1 ; CC(=O)NCCNCc1cccc(-c2nc3cc(OCc4ccccc4)ccc3[nH]2)c1 ; CC(=O)NCCNCc1cccc(-c2nc3cc(C#N)ccc3[nH]2)c1]

The present invention is also disclosed when the X₁, X₂, Z₁, Z₂, Z₃ is C, when Y₁ and Y₂ is N, the synthesis route of the described compound is:

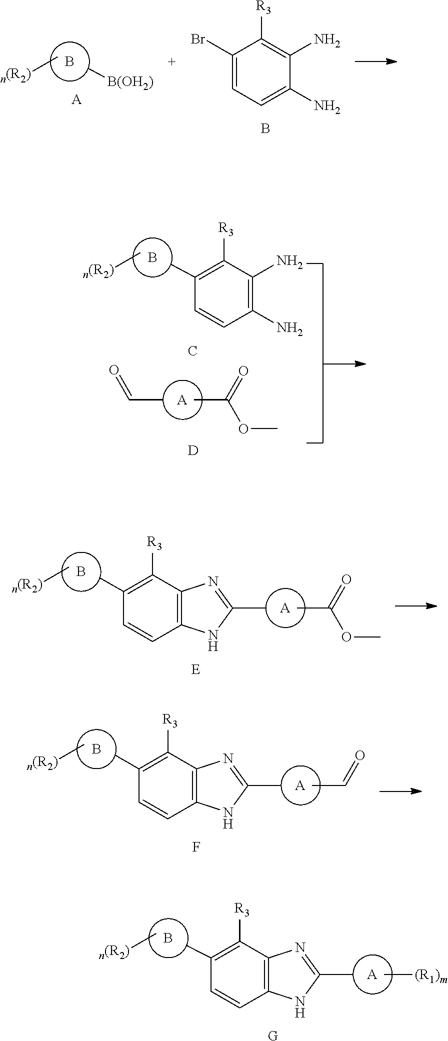

The specific synthesis steps are described as follows:
(1) Compound A and B are coupled under Suzuki coupling; the solvents used include but not limited to: benzene, toluene, ethanol, methanol, 1,4-dioxane, THF, acetone, acetonitrile, hexane, dichloromethane, chloroform, N, N-dimethyl formamide, sulfoxide or mixed solvents optionally composed of these solvents; the bases used include but not limited to: sodium carbonate, potassium carbonate, potassium bicarbonate, sodium bicarbonate, at reaction temperatures of 60° C. to 120° C.; the catalysts used include palladium catalysts such as tetrakis(triphenylphosphine)palladium, dichlorobis(triphenyl-phosphine)Palladium(II);
(2) Compounds C reacted with D to obtain compound E by condensation; the solvents used include but not limited to: benzene, toluene, xylene, p-xylene, o-xylene, m-xylene, ethanol, methanol, 1,4-dioxane, tetrahydrofuran, acetone, acetonitrile, hexane, dichloromethane, chloroform, N, N-dimethylformamide, dimethyl sulfoxide or mixed solvents optionally consisting of these solvents; bases used including but not limited to: sodium bicarbonate, potassium bicarbonate, sodium carbonate, caesium carbonate and potassium carbonate, at a reaction temperature of 40° C. to 130° C.;
(3) Compound F was accomplished by reduction reaction of compound E; the solvents include but not limited to: benzene, toluene, ethanol, methanol, 1,4-dioxane, tetrahydrofuran, acetone, acetonitrile, ethyl acetate, hexane, methyl chloride, chloroform, N, N-dimethyl formamide, dimethyl sulfoxide or mixed solvents consisting of these solvents; the reducing agents include but not limited to: diisobutyl aluminum, sodium borohydride, potassium borohydride, Red-Al and lithium aluminum hydride; the reaction temperature is −78° C. to 120° C.;
(4) Compound G was obtained from compound F by reductive amination reaction; the solvents used include but not limited to: benzene, toluene, ethanol, methanol, 1,4-dioxane, tetrahydrofuran, acetone, acetonitrile, ethyl acetate, hexane, dichloromethane, chloroform, N, N-dimethyl formamide, dimethyl sulfoxide or mixed solvents consisting of these solvents; the reducing agents used include but not limited to: sodium borohydride, potassium borohydride, sodium cyanoborohydride, sodium triacetyl oxorohydride; the reaction temperature is 0 to 60° C.

The present invention also discloses the application of the heterocyclic compounds with pharmaceutically acceptable salts, racemic bodies, rotational optical isomers, or solvent compounds in the preparation of immune checkpoint inhibitors.

The invention also discloses the application of the heterocyclic compounds with pharmaceutically acceptable salts, racemic bodies, rotary photoisomers, or solvent compounds in the preparation of inhibitors with VISTA inhibitory activity.

The invention also discloses the application of the heterocyclic compounds with pharmaceutically acceptable salts, racemic bodies, rotational photoisomers, or solvent compounds in the preparation of antitumor drugs.

The invention also discloses a pharmaceutical composition containing the aforementioned heterocyclic compounds or their pharmaceutically acceptable salt, racemic, rotary optical isomers, or solvent compounds as active ingredients and pharmaceutically acceptable carriers.

The pharmaceutical composition is capsules, tablets, tablets, granules, bolus, injections, syrup, sugar syrup, oral fluids, inhalants, ointments, suppositories or patches.

Beneficial effects: Compared with the prior art, the invention provides a class of immune checkpoint small molecule inhibitors, with novel structure and oral administration, which solves the defects of treatment and resistance of immune checkpoint inhibitors, and is simple to prepare as small molecule inhibitors, convenient for industrial production.

### DETAILED DESCRIPTION OF THE EMBODIMENTS

The invention is further explained in combination with embodiments.

### Example 1

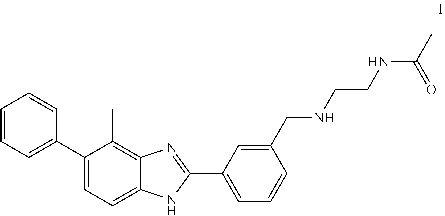 [SMILES: CC(=O)NCCNCc1cccc(-c2nc3c(C)c(-c4ccccc4)ccc3[nH]2)c1]

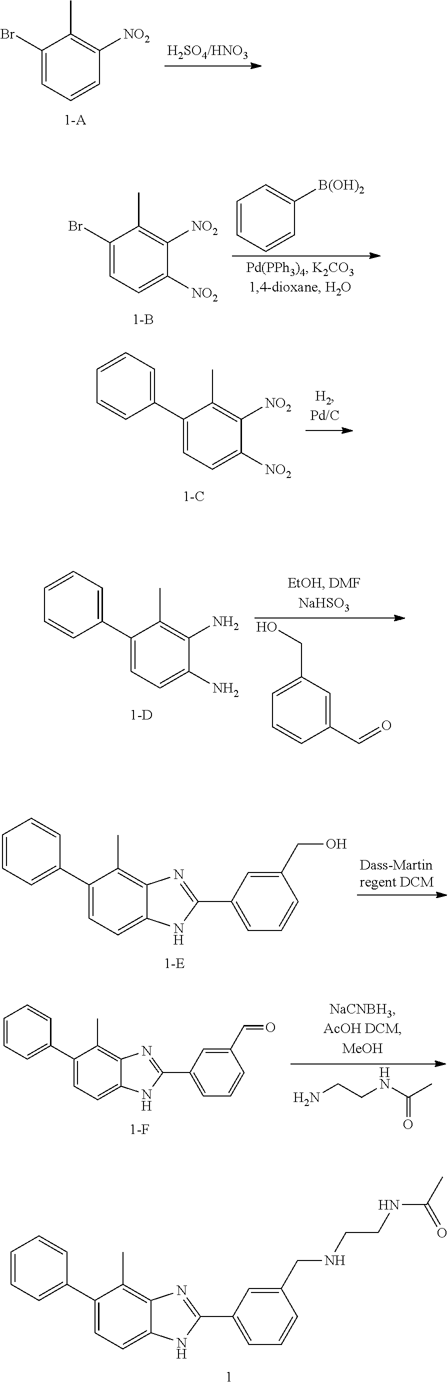

### The Synthesis of Compound 1-B

Compound 1-bromo-2-methyl-3-nitrobenzene (2.5 g) was dissolved in concentrated sulfuric acid (40 mL). In ice bath, concentrated nitric acid (5.1 mL) was added, moved to room temperature and stirred for 2 h. After a TLC detection, the reaction was stopped and the reaction solution was poured into ice water. Filtered, the residue was dissolved with ethyl acetate and was purified by column chromatography (petroleum ether: ethyl acetate=60:1) and compound 1-A (2.5 g) was obtained.

The Synthesis of Compounds 1-C:

Compound 1-B (1.2 g), phenylboric acid (615 mg), tetratriphenylphosphine palladium (174 mg), potassium carbonate (318 mg) were dissolved in 50 mL 1,4-dioxane/water (1:1), and stirred at 80° C. overnight under nitrogen protection. After a TLC detection, the reaction was stopped and the solvent was removed. The residue was dissolved with ethyl acetate, and filtrated via diatomite. The filtrate was concentrated and purified by column chromatography (petroleum ether: ethyl acetate=100:1), compound 1-C was obtained (780 mg).

The Synthesis of Compounds 1-D:

Compound 1-C(450 mg), palladium carbon (45 mg) were dissolved in methanol (15 mL) under atmosphere of H₂. The reaction was stirred at 30° C. overnight. After a TLC detection, the reaction was stopped. The atomite was filtered and the solvent was removed to obtain compound 1-D (350 mg).

### The Synthesis of Compounds 1-E

Compound 3-hydroxymethyl benzaldehyde (248 mg) and sodium bisulfite (182 mg) were dissolved in 10 mL ethanol and the reaction was stirred at room temperature for 2.5 h. compound 1-D (350 mg) was dissolved in DMF (10 mL) and the solvent was dropped into the reaction and stirred at 130° C. for 4 h. After a TLC detection, the solvent was removed and ethyl acetate was added. After extraction, organic phase was concentrated and was purified by column chromatogram (dichloromethane: methanol=50:1), compound 1-E was obtained (189 mg).

### The Synthesis of Compounds 1-F

Compound 1-E (66 mg) was dissolved in DCM (5 mL), Dess-Martin reagent was added (127 mg) in ice bath and stirred at room temperature. After one hour and TLC detection, the reaction was quenched with sodium thiosulfate solution, extracted, and organic phase was concentrated and was purified by column chromatography (dichloromethane: methanol=50:1), and the compound 1-F (60 mg) was obtained.

### Synthesis of Compound 1

Compounds 1-F (30 mg) and N-acetylacethenediamine (20 mg) were dissolved in methanol and dichloromethane (1:1.3 mL), 0.02 mL glacial acetic acid was added. The reaction was stirred for one hour, followed by sodium cyanoboron hydride (31 mg) and stirred for 12 h. After a TLC detection, the solvent was removed and purified by column chromatography (dichloromethane: methanol=20:1) and compound 1 was obtained as white solid. (28 mg). ¹H NMR (300 MHz, Methanol-d₄) δ 8.09 (d, J=6.0 Hz, 1H), 7.86 (s, 1H), 7.61-7.32 (m, 8H), 7.18 (d, J=8.3 Hz, 1H), 3.96 (s, 2H), 3.40 (t, J=3.2 Hz, 2H), 2.83 (t, J=4.4 Hz, 2H), 2.56 (s, 3H), 1.96 (s, 3H).

### Example 2

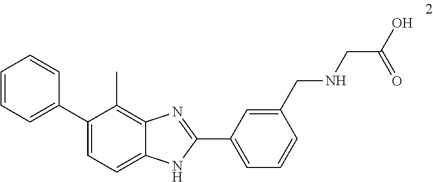 [SMILES: Cc1c(-c2ccccc2)ccc2[nH]c(-c3cccc(CNCC(=O)O)c3)nc12]

Referring to the synthesis method of Example 1, compound 2 can be produced by replacing N-acetylenediamine with glycine. ¹H NMR (300 MHz, Methanol-d₄) δ 7.68 (dd, J=7.5, 2.0 Hz, 1H), 7.64-7.55 (m, 3H), 7.51 (s, 1H), 7.49 (dt, J=2.0, 1.0 Hz, 1H), 7.38-7.27 (m, 4H), 7.12 (t, J=7.5 Hz, 1H), 3.91 (s, 2H), 3.57 (t, J=6.4 Hz, 2H), 2.41 (s, 3H).

### Example 3

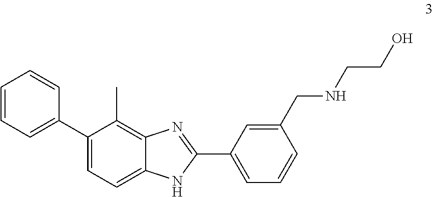 [SMILES: Cc1c(-c2ccccc2)ccc2[nH]c(-c3cccc(CNCCO)c3)nc12]

Referring to the synthesis method of Example 1, compound 3 can be produced by replacing N-acetylethyldiamine with ethanolamine. ¹H NMR (300 MHz, Methanol-d₄) δ 8.21 (d, J=5.0 Hz, 1H), 7.89-7.65 (m, 3H), 7.59 (s, 1H), 7.45-7.29 (m, 5H), 7.15 (t, J=7.5 Hz, 1H), 3.95 (s, 2H), 3.49 (t, J=6.4 Hz, 2H), 2.89 (J=5.5 Hz, 2H), 2.40 (s, 3H).

### Example 4

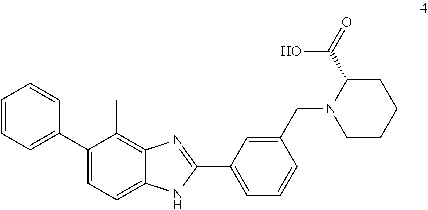 [SMILES: Cc1c(-c2ccccc2)ccc2[nH]c(-c3cccc(CN4CCCC[C@H]4C(=O)O)c3)nc12]

Referring to the synthesis method of Example 1, the N-acetylenediamine is replaced with L-2-piperinic acid to produce compound 4. ¹H NMR (300 MHz, Methanol-d₄) δ 7.67-7.63 (m, 2H), 7.62 (s, 1H), 7.57 (m, 1H), 7.47 (s, 1H), 7.40 (dt, J=2.0, 1.0 Hz, 1H), 7.36-7.28 (m, 4H), 7.11 (t, J=7.5 Hz, 1H), 3.69 (s, 2H), 2.56 (d, J=7.5 Hz, 2H), 2.39 (s, 3H), 1.91-1.45 (m, 6H).

### Example 5

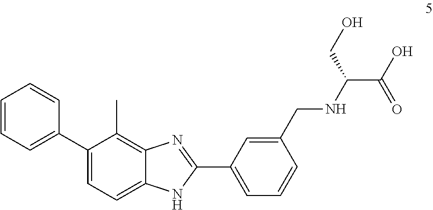 [SMILES: Cc1c(-c2ccccc2)ccc2[nH]c(-c3cccc(CN[C@H](CO)C(=O)O)c3)nc12]

Referring to the synthesis method of Example 1, compound 5 can be produced by replacing N-acetylethylenediamine with L-serine. ¹H NMR (300 MHz, Methanol-d₄) δ 7.68 (d, J=4.3 Hz, 1H), 7.62 (s, 1H), 7.56-7.52 (m, 2H), 7.51 (s, 1H), 7.49-7.47 (m, 1H), 7.38-7.25 (m, 5H), 4.23-4.19 (m, 1H), 4.08-4.05 (m, 2H), 3.97 (s, 2H), 2.40 (s, 3H).

### Example 6

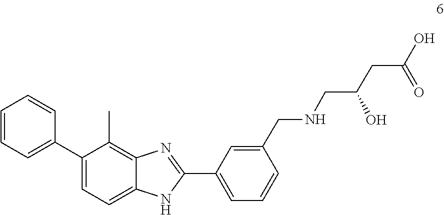 [SMILES: Cc1c(-c2ccccc2)ccc2[nH]c(-c3cccc(CNC[C@@H](O)CC(=O)O)c3)nc12]

Referring to the synthesis method of Example 1, compound 6 can be produced by replacing N with (s)-(+)-4-amino-3 hydroxybutyric acid. ¹H NMR (300 MHz, Methanol-d₄) δ 7.85 (dt, J=7.5, 2.0 Hz, 1H), 7.64-7.58 (m, 3H), 7.47 (s, 1H), 7.47-7.42 (m, 2H), 7.37-7.28 (m, 3H), 7.12 (t, J=7.3 Hz, 1H), 3.99 (d, J=4.9 Hz, 1H), 3.84 (s, 2H), 3.02-2.77 (m, 2H), 2.55-2.43 (m, 2H), 2.40 (s, 3H).

### Example 7

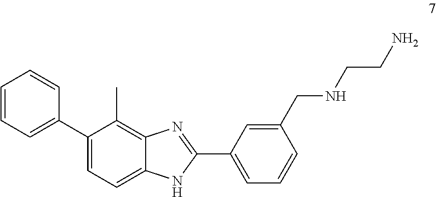 [SMILES: Cc1c(-c2ccccc2)ccc2[nH]c(-c3cccc(CNCCN)c3)nc12]

Referring to the synthesis method of Example 1, compound 7 can be produced by replacing the para-amino benzyl alcohol with ethylenediamine. ¹H NMR (300 MHz, Methanol-d₄) δ 7.80 (d, J=3.8 Hz, 2H), 7.66 (s, 1H), 7.56 (dd, J=7.5, 2.0 Hz, 2H), 7.51 (s, 1H), 7.42-7.27 (m, 4H), 7.06 (d, J=7.4 Hz, 1H), 3.75 (s, 2H), 2.74 (t, J=5.3 Hz, 2H), 2.66 (t, J=7.4 Hz, 2H), 2.41 (s, 3H).

### Example 8

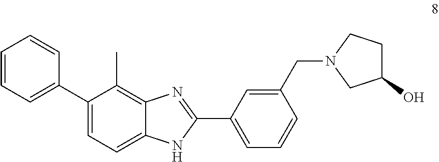 [SMILES: Cc1c(-c2ccccc2)ccc2[nH]c(-c3cccc(CN4CC[C@@H](O)C4)c3)nc12]

Referring to the synthesis method of Example 1, compound 8 can be produced by replacing (R-)-3-pyrrolidine alcohol. ¹H NMR (300 MHz, Methanol-d₄) δ 7.81 (d, J=5.1 Hz, 1H), 7.62 (s, 1H), 7.53-7.45 (m, 3H), 7.43 (s, 1H), 7.39-7.27 (m, 4H), 7.11 (t, J=7.5 Hz, 1H), 3.85 (d, J=4.9 Hz, 1H), 3.63 (s, 2H), 3.23 (d, J=9.5 Hz, 1H), 2.91-2.74 (m, 3H), 2.40 (s, 3H), 1.81 (d, J=22.3 Hz, 2H).

### Example 9

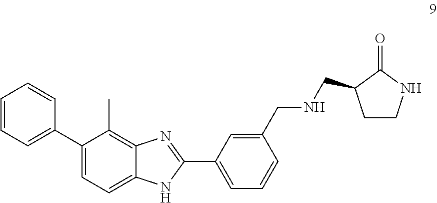 [SMILES: Cc1c(-c2ccccc2)ccc2[nH]c(-c3cccc(CNC[C@@H]4CCNC4=O)c3)nc12]

Referring to the synthesis method of Example 1, compound 9 can be produced by replacing N-acetylenediamine with (S)-3-hydroxymethyl-pyrrolidinone. ¹H NMR (300 MHz, Methanol-d₄) δ 7.80-7.73 (m, 2H), 7.66 (dt, J=1.8, 0.9 Hz, 1H), 7.57-7.51 (m, 2H), 7.47 (s, 1H), 7.39-7.27 (m, 3H), 7.16-7.07 (m, 2H), 3.83 (s, 2H), 3.47-3.25 (m, 2H), 3.23-3.09 (m, 2H), 2.87 (s, 1H), 2.40 (s, 3H), 1.90 (d, J=1.2 Hz, 2H).

### Example 10

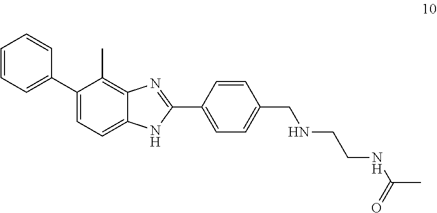 [SMILES: CC(=O)NCCNCc1ccc(-c2nc3c(C)c(-c4ccccc4)ccc3[nH]2)cc1]

Referring to the synthesis method in Example 1, compound 10 can be produced by replacing 3-hydroxymethyl benzaldehyde with p-hydroxymethyl benzaldehyde. ¹H NMR (300 MHz, Methanol-d₄) δ 7.88-7.80 (m, 3H), 7.67-7.51 (m, 4H), 7.44-7.14 (m, 5H), 3.85 (s, 2H), 3.36 (t, J=4.2 Hz, 2H), 2.76 (t, J=5.0 Hz, 2H), 2.40 (s, 3H), 1.90 (s, 3H).

### Example 11

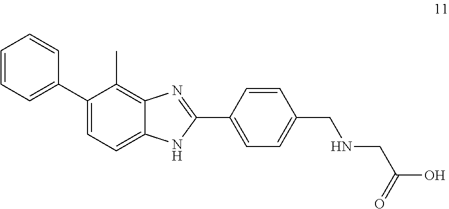 [SMILES: Cc1c(-c2ccccc2)ccc2[nH]c(-c3ccc(CNCC(=O)O)cc3)nc12]

11 In reference to Example 10, compound 11 can be produced by replacing N-acetylenediamine with glycine. ¹H NMR (300 MHz, Methanol-d₄) δ 7.95-7.92 (m, 2H), 7.63-7.58 (m, 4H), 7.38-7.28 (m, 3H), 7.21 (dt, J=7.4, 1.1 Hz, 2H), 3.88 (s, 2H), 3.67 (s, 2H), 2.41 (s, 3H).

### Example 12

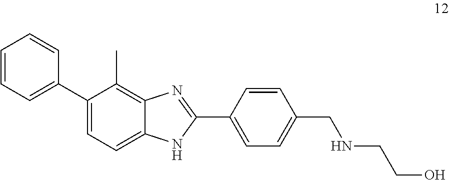 [SMILES: Cc1c(-c2ccccc2)ccc2[nH]c(-c3ccc(CNCCO)cc3)nc12]

Compound 12 can be produced according to the synthesis method of Example 10 by replacing N-acetylethylenediamine with ethanolamine. ¹H NMR (300 MHz, Methanol-d₄) δ 7.95-7.83 (m, 2H), 7.66 (s, 1H), 7.62-7.52 (m, 3H), 7.43-7.29 (m, 3H), 7.20 (dt, J=7.6, 1.1 Hz, 2H), 3.73 (s, 2H), 3.58 (t, J=5.0 Hz, 2H), 2.93 (t, J=6.1 Hz, 2H), 2.41 (s, 3H).

### Example 13

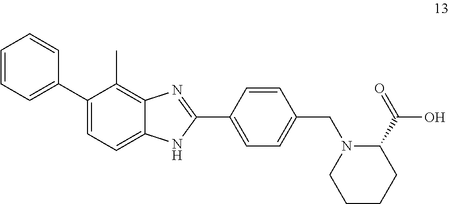 [SMILES: Cc1c(-c2ccccc2)ccc2[nH]c(-c3ccc(CN4CCCC[C@H]4C(=O)O)cc3)nc12]

Referring to the synthesis method of Example 10, the compound 13 can be produced by replacing the N-acetylethylenediamine with L-2-piperidinoic acid. ¹H NMR (300 MHz, Methanol-d₄) δ 7.89-7.78 (m, 2H), 7.73-7.60 (m, 3H), 7.55 (s, 1H), 7.39-7.25 (m, 3H), 7.00 (dt, J=7.5, 1.0 Hz, 2H), 4.33 (s, 1H), 3.73 (s, 2H), 2.61 (d, J=15.0 Hz, 2H), 2.39 (s, 3H), 1.98-1.46 (m, 6H).

### Example 14

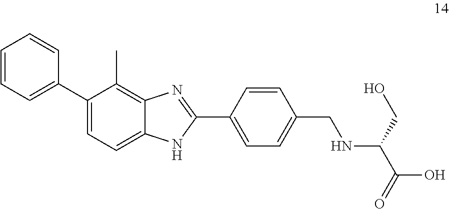 [SMILES: Cc1c(-c2ccccc2)ccc2[nH]c(-c3ccc(CN[C@H](CO)C(=O)O)cc3)nc12]

Referring to the synthesis method of Example 10, the compound 14 is produced by replacing N-acetylethylenediamine with L-serine. ¹H NMR (300 MHz, Methanol-d₄) δ 7.93-7.83 (m, 2H), 7.67-7.50 (m, 4H), 7.43-7.26 (m, 3H), 7.16 (dt, J=7.5, 1.0 Hz, 2H), 4.25 (s, 1H), 4.03-3.86 (m, 3H), 3.78 (d, J=5.5 Hz, 1H), 2.40 (s, 3H).

### Example 15

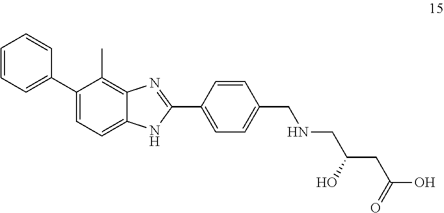 [SMILES: Cc1c(-c2ccccc2)ccc2[nH]c(-c3ccc(CNC[C@@H](O)CC(=O)O)cc3)nc12]

Referring to the synthesis method of Example 10, compound 15 can be produced by replacing (s)-(+)-4 amino-3 hydroxybutyric acid. ¹H NMR (300 MHz, Methanol-d₄) δ 7.89-7.78 (m, 2H), 7.69-7.51 (m, 4H), 7.44-7.26 (m, 3H), 7.26-7.10 (m, 2H), 3.96-3.81 (m, 3H), 3.29-3.06 (m, 2H), 2.52-2.42 (m, 2H), 2.40 (s, 3H).

### Example 16

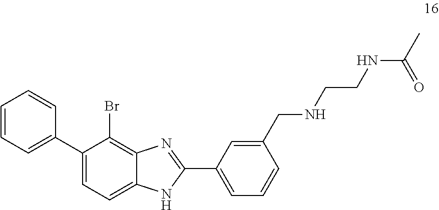 [SMILES: CC(=O)NCCNCc1cccc(-c2nc3c(Br)c(-c4ccccc4)ccc3[nH]2)c1]

Referring to the synthesis method of Example 1, replace 1-A with 1-B-1-iodine-3-nitrobenzene. ¹H NMR (300 MHz, Methanol-d₄) δ 7.81-7.75 (m, 2H), 7.69-7.63 (m, 2H), 7.51 (s, 1H), 7.46 (dt, J=2.0, 1.0 Hz, 1H), 7.39-7.27 (m, 4H), 7.12 (t, J=7.5 Hz, 1H), 3.80 (s, 2H), 3.36 (t, J=3.5 Hz, 2H), 2.73 (t, J=3.5 Hz, 2H), 1.95 (s, 3H).

### Example 17

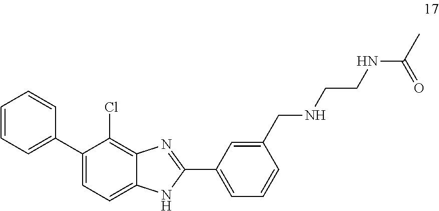 [SMILES: CC(=O)NCCNCc1cccc(-c2nc3c(Cl)c(-c4ccccc4)ccc3[nH]2)c1]

Referring to the synthesis method of Example 1, replace 1-A with 2-chlorine-1-bromine-3-nitrobenzene. ¹H NMR (300 MHz, Methanol-d₄) δ 7.97 (s, 1H), 7.81-7.75 (m, 2H), 7.58 (dt, J=7.0, 1.5 Hz, 2H), 7.52-7.48 (m, 2H), 7.41 (dt, J=7.5, 1.0 Hz, 1H), 7.36-7.28 (m, 3H), 7.12 (t, J=7.5 Hz, 1H), 3.80 (s, 2H), 3.35 (t, J=7.5 Hz, 2H), 2.73 (t, J=6.5 Hz, 2H), 1.90 (s, 3H).

### Example 18

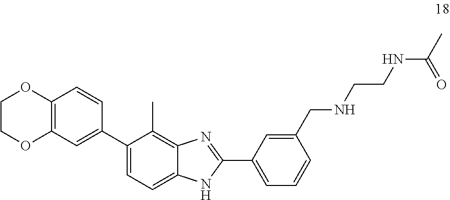 [SMILES: CC(=O)NCCNCc1cccc(-c2nc3c(C)c(-c4ccc5c(c4)OCCO5)ccc3[nH]2)c1]

Referring to the synthesis method of Example 1, the compound 18 can be produced by replacing the phenylboric acid with benzo-1-4-dioxane-6-boric acid. ¹H NMR (300 MHz, Methanol-d₄) δ 7.48-7.42 (m, 3H), 7.41 (s, 1H), 7.36 (s, 1H), 7.34-7.29 (m, 1H), 7.15 (d, J=2.0 Hz, 1H), 7.09 (t, J=7.8 Hz, 1H), 6.91 (d, J=7.5 Hz, 1H), 4.31 (d, J=10.4 Hz, 4H), 3.82 (s, 2H), 3.35 (t, J=4.8 Hz, 2H), 2.73 (t, J=7.1 Hz, 2H), 2.38 (s, 3H), 1.93 (s, 3H).

### Example 19

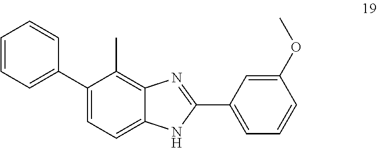 [SMILES: COc1cccc(-c2nc3c(C)c(-c4ccccc4)ccc3[nH]2)c1]

Referring to the synthesis method of Example 1, replacement of 3-hydroxymethyl benzaldehyde with 3-methoxybenzaldehyde yields compound 19. ¹H NMR (300 MHz, Methanol-d₄) δ 7.71 (s, 1H), 7.68 (d, J=7.5 Hz, 1H), 7.61 (dd, J=7.6, 2.0 Hz, 2H), 7.40-7.28 (m, 5H), 7.05 (t, J=7.5 Hz, 1H), 6.91-6.85 (m, 1H), 3.87 (s, 3H), 2.47 (s, 3H).

### Example 20

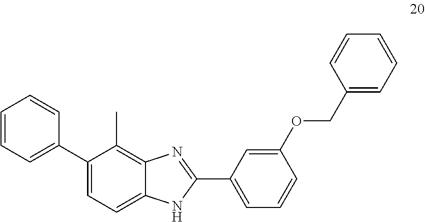 [SMILES: Cc1c(-c2ccccc2)ccc2[nH]c(-c3cccc(OCc4ccccc4)c3)nc12]

Referring to the synthesis method of Example 1, replacement of 3-hydroxymethyl benzaldehyde with 3-benzyl methoxybenzaldehyde yields compound 20.1H NMR (300 MHz, Methanol-d4) δ 7.66 (s, 1H), 7.64-7.59 (m, 2H), 7.44-7.27 (m, 11H), 7.06 (t, J=7.4 Hz, 1H), 6.90 (dt, J=7.5, 2.0 Hz, 1H), 5.14 (t, J=1.0 Hz, 2H), 2.43 (s, 3H).

### Example 21

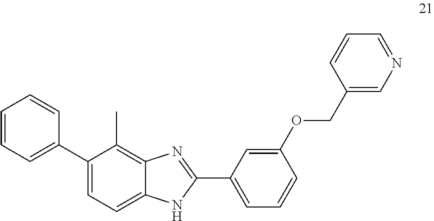 [SMILES: Cc1c(-c2ccccc2)ccc2[nH]c(-c3cccc(OCc4cccnc4)c3)nc12]

Referring to the synthesis method of Example 1, compound 21 is produced by replacing 3-hydroxymethyl benzaldehyde with 3-(pyridine-3-methoxy) benzaldehyde. ¹H NMR (300 MHz, Methanol-d₄) δ 8.61-8.50 (m, 2H), 7.66 (s, 1H), 7.64-7.57 (m, 2H), 7.52 (dt, J=8.1, 1.3 Hz, 1H), 7.45-7.25 (m, 7H), 7.06 (t, J=7.4 Hz, 1H), 6.87 (dt, J=7.5, 2.0 Hz, 1H), 5.28 (s, 2H), 2.43 (s, 3H).

### Example 22

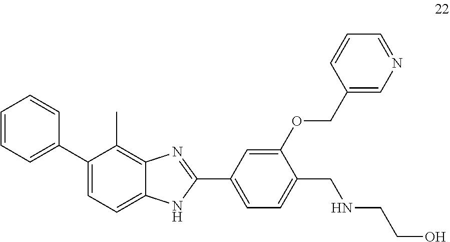 [SMILES: Cc1c(-c2ccccc2)ccc2[nH]c(-c3ccc(CNCCO)c(OCc4cccnc4)c3)nc12]

Referring to the synthesis method of Example 12, compound 22 is produced by replacing 3-(pyridine-3-methoxy)-4-hydroxymethyl benzaldehyde. ¹H NMR (300 MHz, Methanol-d 4) δ 8.67-8.42 (m, 2H), 7.64-7.55 (m, 3H), 7.50 (s, 1H), 7.43 (s, 1H), 7.40-7.25 (m, 5H), 7.21 (d, J=2.0 Hz, 1H), 7.00 (dt, J=7.5, 1.0 Hz, 1H), 5.13 (s, 2H), 3.94 (s, 2H), 3.60 (t, J=4.4 Hz, 2H), 3.18 (t, J=3.9 Hz, 2H), 2.41 (s, 3H).

### Example 23

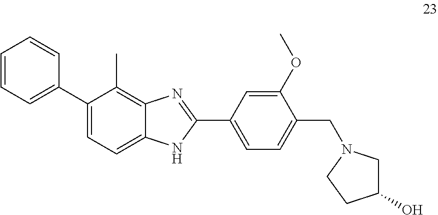 [SMILES: COc1cc(-c2nc3c(C)c(-c4ccccc4)ccc3[nH]2)ccc1CN1CC[C@@H](O)C1]

Referring to the synthesis method of Example 8, compound 23 can be produced by replacing 3-hydroxymethyl benzaldehyde with 3-methoxy-4-hydroxymethyl benzaldehyde. ¹H NMR (300 MHz, Methanol-d 4) δ 7.66 (s, 1H), 7.64-7.59 (m, 2H), 7.39-7.25 (m, 5H), 7.19 (d, J=2.0 Hz, 1H), 6.99 (dt, J=7.4, 1.0 Hz, 1H), 3.91 (s, 2H), 3.77 (s, 3H), 3.72-3.56 (m, 2H), 3.20 (t, J=7.5 Hz, 2H), 2.76 (t, J=5.3 Hz, 2H), 2.43 (s, 3H), 1.83 (d, J=5.2 Hz, 2H).

### Example 24

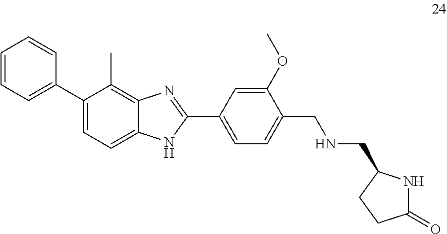 [SMILES: COc1cc(-c2nc3c(C)c(-c4ccccc4)ccc3[nH]2)ccc1CNC[C@@H]1CCC(=O)N1]

Referring to the synthesis method of Example 9, replacement of 3-hydroxymethyl benzaldehyde with 3-methoxyl-4-hydroxymethyl benzaldehyde produces compound 24. ¹H NMR (300 MHz, Methanol-d 4) δ 7.70-7.53 (m, 3H), 7.43 (s, 1H), 7.39-7.15 (m, 5H), 7.00 (dt, J=7.6, 1.0 Hz, 1H), 4.13-3.96 (m, 3H), 3.81 (s, 3H), 3.07 (t, J=4.7 Hz, 1H), 2.56 (t, J=5.5 Hz, 1H), 2.51-2.41 (m, 3H), 2.39 (s, 1H), 1.84 (d, J=4.7 Hz, 2H).

### Example 25

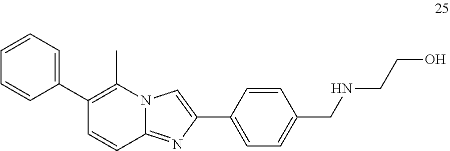 [SMILES: Cc1c(-c2ccccc2)ccc2nc(-c3ccc(CNCCO)cc3)cn12]

synthetic method

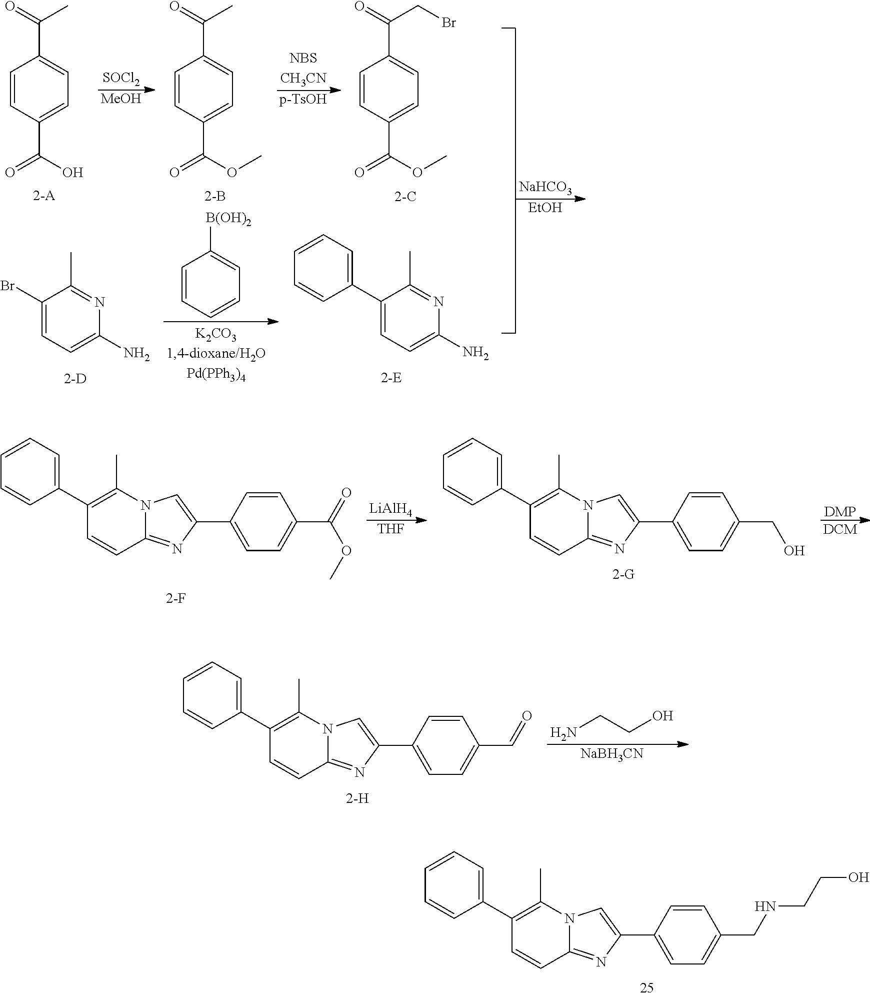

### The Synthesis of Compound 2-B, Respectively

At 0° C., 2-A (821 mg) was dissolved in 10 mL methanol and dropped it into sulfoxide chloride (0.725 mL). After the addition, the reaction was stirred at 50° C. After 3 hours and a TLC detection, the reaction was completed and was concentrated, and the compound 2-B (830 mg) was purified by column chromatography (petroleum ether: ethyl acetate=15:1).

### The Synthesis of Compounds 2-C

Compound 2-B (510 mg) was dissolved with toluene sulfonic acid (816 mg) in dichloromethane, NBS (509 mg) was added in batches and stirred at 90° C. for 8 h. After a TLC detection, the solvent was removed and DCM was added in. Washed with H₂O twice, saturated salt washed once, organic phase was concentrated to obtain compound 2-C(530 mg).

### The Synthesis of Compounds 2-E

Compound 2-D (374 mg), phenylboric acid (292 mg), potassium carbonate (415 mg) and tetritrophenylphosphine palladium (70 mg) were dissolved in 10 mL dioxane/water (1:1), protected by nitrogen gas, and stirred at 80° C. overnight. After a TLC detection, the solvent was removed and purified by column chromatography (petroleum ether ethyl acetate=1:1) to obtain compound 2-E (350 mg).

### The Synthesis of Compound 2-F

Compound 2-C(480 mg), 2-E (416 mg) and sodium bicarbonate (320 mg) were dissolved in ethanol and stirred at 85° C. overnight. After a TLC detection, the reaction was filtered and the filter was washed by ethanol. Compound 2-F was obtained (336 mg).

### The Synthesis of Compound 2-G

Add THF into LiAlH₄ (57 mg), then 2-F (336 mg) was added in and the reaction was stirred at room temperature for two hours. After a TLC detection, the reaction was quenched by 0.5 mL of sodium hydroxide solution, then filtered via diatomite, compound 2-G (260 mg) was obtained via purification by the column chromatography (dichloromethane: methanol=30:1).

### The Synthesis of Compound 2-H

Compound 2-G (100 mg) was dissolved in dichloromethane, and Dess-Martin reagent (204 mg) was added in. After 0.5 h, the reaction was quenched with sodium thiosulfate solution, the organic phase was concentrated and purified by column (dichloromethane: methanol=80:1), and compound 2-H (95 mg) was obtained.

### Synthesis of Compound 25

2-H (50 mg) and ethanolamine (20 mg) were dissolved in dichloromethane/methanol (1:1), a drop of acetic acid was added in and the reaction was stirred for 0.5 h. Then sodium cyanoborohydride (20 mg) was added in. After 5 hours, the solvent was removed and ethyl acetate was added in, washed with saturated sodium bicarbonate solution. The organic phase was concentrated and purified by column chromatography (dichloromide: methanol=15:1) to obtain compound 25 (40 mg). ¹H NMR (300 MHz, Methanol-d4) δ 8.26 (d, J=2.7 Hz, 1H), 7.96 (s, 1H), 7.67-7.43 (m, 3H), 7.40-7.20 (m, 7H), 3.71 (s, 2H), 3.58 (t, J=4.9 Hz, 2H), 2.93 (t, J=3.7 Hz, 2H), 2.49 (s, 3H).

### Example 26

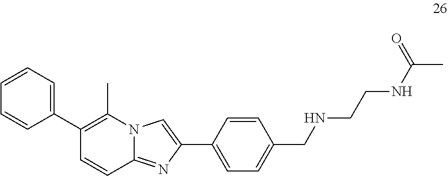 [SMILES: CC(=O)NCCNCc1ccc(-c2cn3c(C)c(-c4ccccc4)ccc3n2)cc1]

Referring to the synthesis method of Example 25, compound 26 can be replaced with N-acetylenediamine. ¹H NMR (300 MHz, Methanol-d4) δ 8.28 (t, J=5.9 Hz, 1H), 7.78 (s, 1H), 7.59 (d, J=2.5 Hz, 1H), 7.52-7.43 (m, 4H), 7.37-7.24 (m, 5H), 3.83 (s, 2H), 3.36 (t, J=4.2 Hz, 2H), 2.76 (t, J=3.8 Hz, 2H), 2.49 (s, 3H), 1.90 (s, 3H).

### Example 27

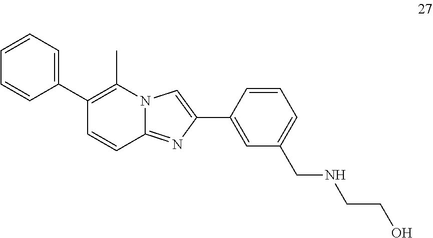 [SMILES: Cc1c(-c2ccccc2)ccc2nc(-c3cccc(CNCCO)c3)cn12]

With reference to the synthesis method of Example 25, replace methyl para-formate with benzaldehyde 3-methyl formate. ¹H NMR (300 MHz, Methanol-d4) δ 8.20 (t, J=6.1 Hz, 1H), 7.96 (s, 1H), 7.84-7.63 (m, 2H), 7.59 (d, J=4.8 Hz, 2H), 7.30-7.13 (m, 6H), 3.89-3.71 (m, 2H), 3.59 (t, J=5.1 Hz, 2H), 2.81 (t, J=4.4 Hz, 2H), 2.49 (s, 3H).

### Example 28

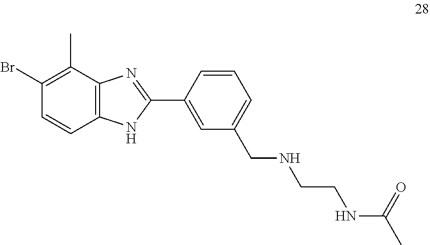 [SMILES: CC(=O)NCCNCc1cccc(-c2nc3c(C)c(Br)ccc3[nH]2)c1]

Referring to the synthesis method of Example 10, the compound 28 can be produced. ¹H NMR (300 MHz, Methanol-d4) δ 7.97-7.90 (m, 2H), 7.84 (s, 1H), 7.47-7.31 (m, 2H), 7.20 (d, J=7.6, 1.1 Hz, 2H), 3.81 (s, 2H), 3.36 (t, J=3.1 Hz, 2H), 2.76 (t, J=4.2 Hz, 2H), 2.40 (s, 3H), 1.89 (s, 3H).

### Example 29

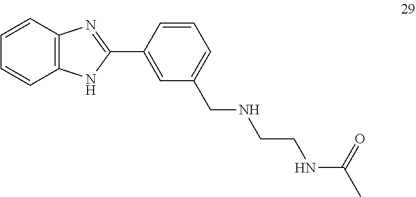 [SMILES: CC(=O)NCCNCc1cccc(-c2nc3ccccc3[nH]2)c1]

Referring to the synthesis method of Example 10, the compound 29 can be produced. ¹H NMR (300 MHz, Methanol-d4) δ 7.91-7.77 (m, 3H), 7.54 (d, J=59.5 Hz, 2H), 7.21 (d, J=7.5 Hz, 2H), 7.14 (d, J=4.8 Hz, 2H), 3.83 (s, 2H), 3.27 (t, J=4.2 Hz, 2H), 2.80 (t, J=3.7 Hz, 2H), 1.93 (s, 3H).

### Example 30

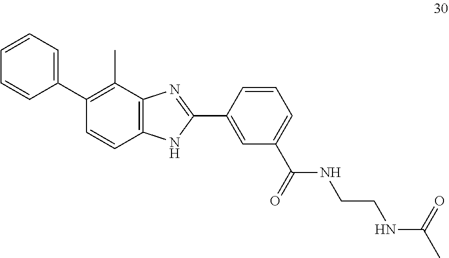 [SMILES: CC(=O)NCCNC(=O)c1cccc(-c2nc3c(C)c(-c4ccccc4)ccc3[nH]2)c1]

Referring to the synthesis method of Example 10, the compound 30 can be produced. ¹H NMR (300 MHz, Methanol-d4) δ 8.40 (t, J=2.0 Hz, 1H), 8.10-7.97 (m, 1H), 7.75-7.47 (m, 6H), 7.43-7.27 (m, 3H), 3.42 (t, J=4.2 Hz, 2H), 3.33 (t, J=3.5 Hz, 2H), 2.40 (s, 3H), 1.91 (s, 3H).

### Example 31

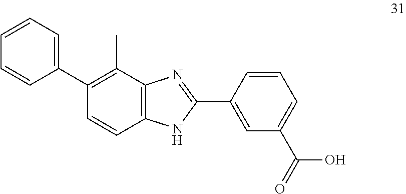 [SMILES: Cc1c(-c2ccccc2)ccc2[nH]c(-c3cccc(C(=O)O)c3)nc12]

Referring to the synthesis method of Example 30, the compound 31 can be produced. ¹H NMR (300 MHz, Methanol-d4) δ 8.05 (t, J=2.0 Hz, 1H), 7.96 (dt, J=7.5, 2.0 Hz, 1H), 7.71 (s, 1H), 7.68 (dt, J=7.5, 2.0 Hz, 1H), 7.63-7.57 (m, 3H), 7.50 (t, J=7.5 Hz, 1H), 7.40-7.27 (m, 3H), 2.41 (s, 3H).

### Example 32

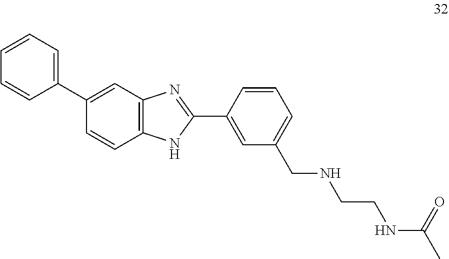 [SMILES: CC(=O)NCCNCc1cccc(-c2nc3cc(-c4ccccc4)ccc3[nH]2)c1]

Referring to the synthesis method of Example 10, the compound 32 can be produced. ¹H NMR (300 MHz, Methanol-d4) δ 7.86-7.81 (m, 3H), 7.72 (d, J=20.7 Hz, 2H), 7.56-7.52 (m, 2H), 7.41-7.36 (m, 2H), 7.34-7.28 (m, 1H), 7.20 (dt, J=7.6, 1.1 Hz, 2H), 3.90 (s, 2H), 3.32 (t, J=3.7 Hz, 2H), 2.76 (t, J=4.5 Hz, 2H), 1.90 (s, 3H).

### Example 33

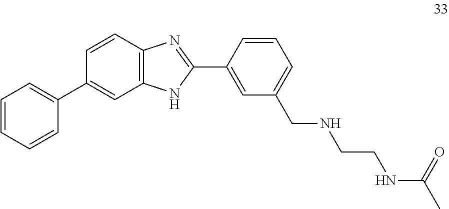 [SMILES: CC(=O)NCCNCc1cccc(-c2nc3ccc(-c4ccccc4)cc3[nH]2)c1]

Referring to the synthesis method of Example 10, the compound 33 can be produced. ¹H NMR (300 MHz, Methanol-d4) δ 7.86-7.81 (m, 4H), 7.56-7.51 (m, 3H), 7.44 (t, J=7.5 Hz, 2H), 7.35 (d, J=7.3 Hz, 1H), 7.20 (dt, J=7.6, 1.1 Hz, 2H), 3.93 (s, 2H), 3.32 (t, J=3.5 Hz, 2H), 2.76 (t, J=4.2 Hz, 2H), 1.90 (s, 3H).

### Example 34

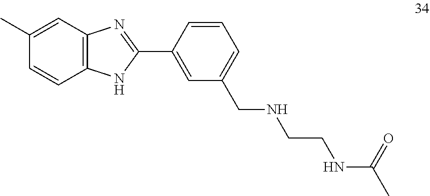 [SMILES: CC(=O)NCCNCc1cccc(-c2nc3cc(C)ccc3[nH]2)c1]

Referring to the synthesis method of Example 10, the compound 34 can be produced. ¹H NMR (300 MHz, Methanol-d4) δ 7.87-7.78 (m, 3H), 7.26 (s, 1H), 7.19 (dt, J=7.5, 1.0 Hz, 2H), 7.06 (s, 1H), 3.34 (s, 2H), 2.76 (s, 2H), 2.46 (s, 3H), 1.93 (s, 3H).

### Example 35

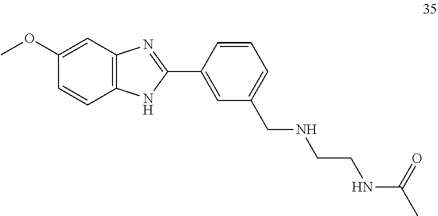 [SMILES: COc1ccc2[nH]c(-c3cccc(CNCCNC(C)=O)c3)nc2c1]

Referring to the synthesis method of the Example 10, the compound 35 can be produced. ¹H NMR (300 MHz, Methanol-d4) δ 7.88-7.71 (m, 3H), 7.49 (s, 1H), 7.14 (dt, J=7.4, 1.0 Hz, 2H), 6.85 (s, 1H), 3.84 (s, 3H), 3.71 (t, J=3.4 Hz, 2H), 3.32 (t, J=7.4, 1.0 Hz, 2H), 2.76 (s, 2H), 1.89 (s, 3H).

### Example 36

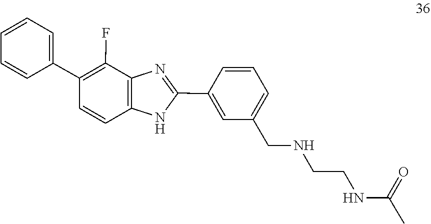 [SMILES: CC(=O)NCCNCc1cccc(-c2nc3c(F)c(-c4ccccc4)ccc3[nH]2)c1]

Referring to the synthesis method of Example 10, the compound 36 can be produced. ¹H NMR (300 MHz, Methanol-d4) δ 7.96-7.90 (m, 2H), 7.83 (d, J=4.9 Hz, 2H), 7.71-7.65 (m, 2H), 7.37-7.29 (m, 3H), 7.16 (dt, J=7.5, 1.1 Hz, 2H), 3.84 (s, 2H), 3.36 (t, J=4.2 Hz, 2H), 2.76 (t, J=3.9 Hz, 2H), 1.90 (s, 3H).

### Example 37

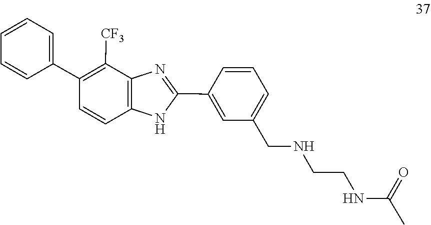 [SMILES: CC(=O)NCCNCc1cccc(-c2nc3c(C(F)(F)F)c(-c4ccccc4)ccc3[nH]2)c1]

Referring to the synthesis method of Example 10, the compound 36 can be produced. ¹H NMR (300 MHz, Methanol-d4) δ 7.89-7.49 (m, 8H), 7.48-7.36 (m, 3H), 7.16-7.07 (m, 1H), 3.80 (s, 2H), 3.35 (t, J=3.3 Hz, 2H), 2.73 (t, J=4.2 Hz, 2H), 1.91 (s, 3H).

### Example 38

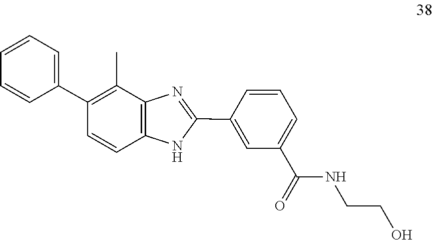 [SMILES: Cc1c(-c2ccccc2)ccc2[nH]c(-c3cccc(C(=O)NCCO)c3)nc12]

Referring to the synthesis method of Example 30, the compound 38 can be produced. ¹H NMR (300 MHz, Methanol-d4) δ 8.40 (t, J=2.0 Hz, 1H), 7.94-7.86 (m, 2H), 7.67-7.60 (m, 3H), 7.57 (dt, J=7.5, 2.1 Hz, 1H), 7.51 (t, J=7.5 Hz, 1H), 7.39-7.27 (m, 3H), 3.49 (t, J=3.0 Hz, 2H), 3.39 (t, J=4.5 Hz, 2H), 2.41 (s, 3H).

### Example 39

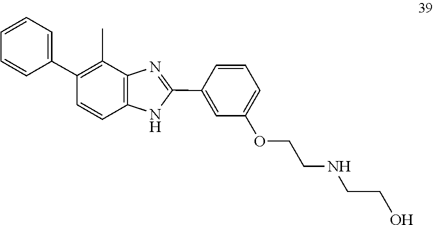 [SMILES: Cc1c(-c2ccccc2)ccc2[nH]c(-c3cccc(OCCNCCO)c3)nc12]

Referring to the synthesis method of Example 1, the compound 39 can be produced. ¹H NMR (300 MHz, Methanol-d4) δ 7.68-7.58 (m, 3H), 7.47-7.36 (m, 3H), 7.36-7.28 (m, 3H), 7.06 (t, J=7.4 Hz, 1H), 6.94-6.88 (m, 1H), 4.08 (t, J=3.2 Hz, 2H), 3.57 (t, J=4.9 Hz, 2H), 2.94 (t, J=2.6 Hz, 2H), 2.75 (t, J=3.1 Hz, 2H), 2.43 (s, 3H).

### Example 40

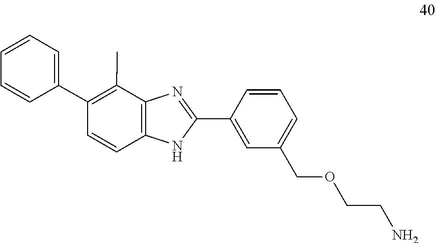 [SMILES: Cc1c(-c2ccccc2)ccc2[nH]c(-c3cccc(COCCN)c3)nc12]

Referring to the synthesis method of Example 1, the compound 40 can be produced. ¹H NMR (300 MHz, Methanol-d4) δ 7.71-7.47 (m, 7H), 7.41-7.28 (m, 3H), 7.16 (t, J=7.5 Hz, 1H), 4.48 (s, 2H), 3.51 (t, J=2.5 Hz, 2H), 2.69 (t, J=3.2 Hz, 2H), 2.41 (s, 3H).

### Example 41

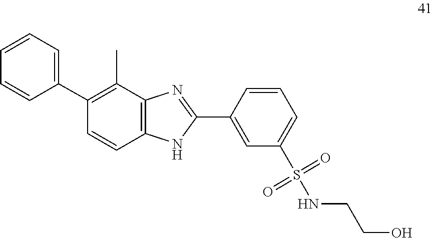 [SMILES: Cc1c(-c2ccccc2)ccc2[nH]c(-c3cccc(S(=O)(=O)NCCO)c3)nc12]

Referring to the synthesis method of Example 1, the compound 41 can be produced. ¹H NMR (300 MHz, Methanol-d4) δ 7.92-7.80 (m, 2H), 7.69-7.54 (m, 6H), 7.37-7.28 (m, 3H), 3.57 (t, J=4.9 Hz, 2H), 3.12 (t, J=3.1 Hz, 2H), 2.40 (s, 3H).

### Example 42

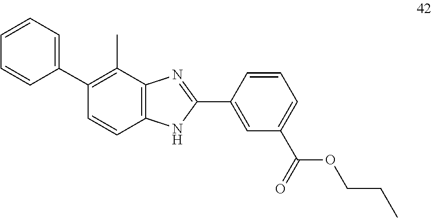 [SMILES: CCCOC(=O)c1cccc(-c2nc3c(C)c(-c4ccccc4)ccc3[nH]2)c1]

Referring to the synthesis method of Example 38, the compound 42 can be produced. ¹H NMR (300 MHz, Methanol-d4) δ 8.11 (t, J=2.0 Hz, 1H), 7.98 (dt, J=7.5, 2.0 Hz, 1H), 7.73-7.59 (m, 5H), 7.53 (t, J=7.5 Hz, 1H), 7.40-7.26 (m, 3H), 4.22 (t, J=3.2, 2H), 2.41 (s, 3H), 1.83-1.63 (m, 2H), 1.03 (t, 3H).

### Example 43

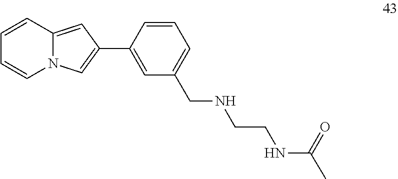 [SMILES: CC(=O)NCCNCc1cccc(-c2cc3ccccn3c2)c1]

synthetic method

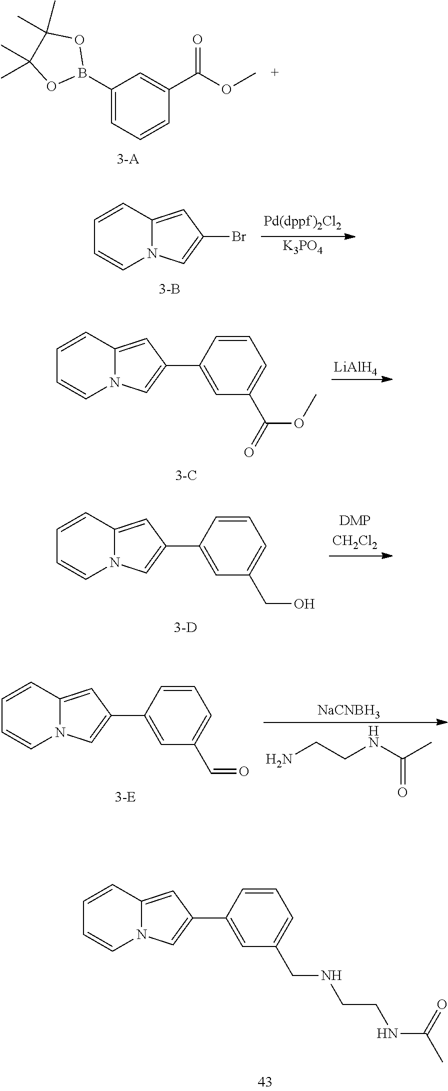

### The Synthesis of Compound 3-C

Compound 3-A (786 mg) was dissolved with 3-B (588 mg) in 1,4-dioxane/water (6 mL, 5:1), Then 1,1′-didiphenylphosphonium palladium dichloride (150 mg) and potassium phosphate (970 mg) was added in and protected by N₂. The reaction was stirred at 90° C. overnight. After a TLC dectection, ethyl acetate was added in and extracted with water. The organic phase was collected and concentrated, and compound 3-C (502 mg) was obtained through purification by the column (petroleum ether ethyl acetate=20:1).

### The Synthesis of Compounds 3-D

### Synthesis of Reference Compound 2-G, Compound 3-D

### The Synthesis of Compounds 3-E

### Synthesis of Reference Compound 2-H, Compound 3-E

### Synthesis of Compound 43

Referring to the synthesis method of compound 25, compound 43 can be produced. 1H NMR (300 MHz, Methanol-d 4) δ 8.07 (s, 1H), 7.79 (s, 1H), 7.72-7.64 (m, 2H), 7.55-7.38 (m, 4H), 7.33 (s, 1H), 7.19 (t, J=7.5 Hz, 1H), 3.92-3.62 (m, 2H), 3.27 (s, 2H), 2.80 (s, 2H), 1.93 (s, 3H).

### Example 44

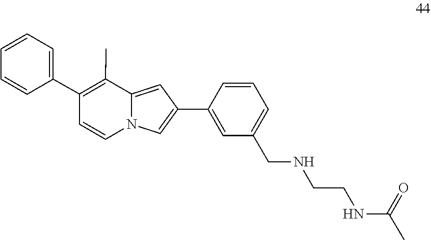 [SMILES: CC(=O)NCCNCc1cccc(-c2cc3c(C)c(-c4ccccc4)ccn3c2)c1]

The reference synthesis method of compound 43 produces the compound 44. ¹H NMR (300 MHz, Methanol-d 4) δ 7.79 (d, J=15.6 Hz, 2H), 7.74-7.62 (m, 3H), 7.49-7.28 (m, 6H), 7.25 (s, 1H), 7.15 (t, J=7.5 Hz, 1H), 3.83 (s, 2H), 3.36 (t, J=3.2 Hz, 2H), 2.88 (t, J=4.5 Hz, 2H), 2.39 (s, 3H), 1.90 (s, 3H).

### Example 45

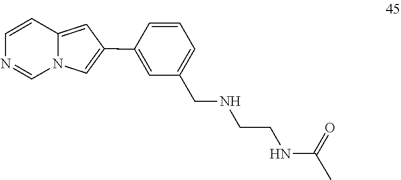 [SMILES: CC(=O)NCCNCc1cccc(-c2cc3ccncn3c2)c1]

Referring to the synthesis method of the compound 43, the compound 45 can be produced. ¹H NMR (300 MHz, Methanol-d 4) δ 8.14 (s, 1H), 7.78 (d, J=27.4 Hz, 2H), 7.61-7.38 (m, 5H), 7.30-7.19 (m, 1H), 3.79 (s, 2H), 3.27 (t, J=2.3 Hz, 2H), 2.80 (t, J=4.1 Hz, 2H), 1.93 (s, 3H).

### Example 46

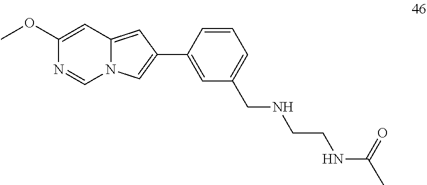 [SMILES: COc1cc2cc(-c3cccc(CNCCNC(C)=O)c3)cn2cn1]

Referring to the synthesis method of compound 43, compound 46 can be produced. ¹H NMR (300 MHz, Methanol-d 4) δ 7.78 (d, J=33.0 Hz, 2H), 7.62-7.26 (m, 5H), 3.94 (s, 3H), 3.81 (s, 2H), 3.32 (t, J=3.1 Hz, 2H), 2.65 (t, J=4.6 Hz, 2H), 1.89 (s, 3H).

### Example 47

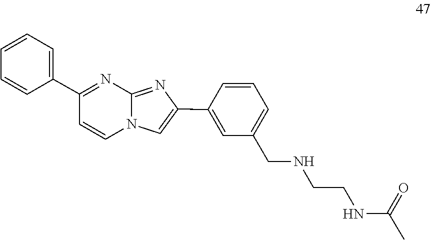 [SMILES: CC(=O)NCCNCc1cccc(-c2cn3ccc(-c4ccccc4)nc3n2)c1]

Referring to the synthesis method of compound 43, compound 47 can be produced. ¹H NMR (300 MHz, Methanol-d 4) δ 8.23 (s, 1H), 8.17-8.09 (m, 2H), 7.84 (s, 1H), 7.72-7.57 (m, 3H), 7.51-7.40 (m, 4H), 3.79 (s, 2H), 3.27 (t, J=2.8 Hz, 2H), 2.73 (t, J=4.2 Hz, 2H), 1.90 (s, 3H).

### Example 48

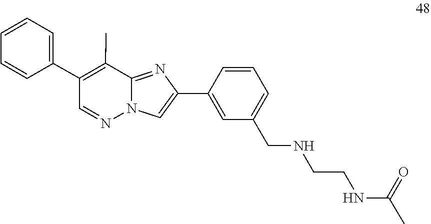 [SMILES: CC(=O)NCCNCc1cccc(-c2cn3ncc(-c4ccccc4)c(C)c3n2)c1]

Reference synthesis of compound 43 yields compound 48. ¹H NMR (300 MHz, Methanol-d 4) δ 7.84 (s, 1H), 7.73-7.63 (m, 3H), 7.46 (dddd, J=8.4, 4.2, 2.1, 1.2 Hz, 3H), 7.38-7.26 (m, 3H), 3.82 (s, 2H), 3.32 (s, J=2.3 Hz, 2H), 2.73 (t, J=3.8 Hz, 2H), 2.36 (s, 3H), 1.79 (s, 3H).

### Example 49

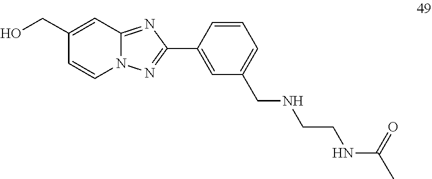 [SMILES: CC(=O)NCCNCc1cccc(-c2nc3cc(CO)ccn3n2)c1]

Reference synthesis of compound 43 yields compound 49. ¹H NMR (300 MHz, Methanol-d 4) δ 7.83 (dt, J=7.5, 2.0 Hz, 1H), 7.79 (s, 1H), 7.56-7.38 (m, 3H), 7.17-7.04 (m, 2H), 4.59 (s, 2H), 3.79 (s, 2H), 3.27 (m, J=3.0 Hz, 2H), 2.76 (t, J=3.9 Hz, 2H), 1.89 (s, 3H).

### Example 50

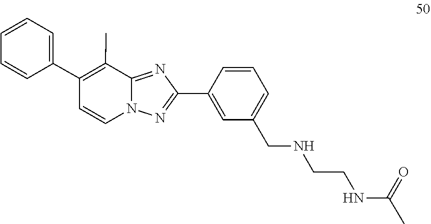 [SMILES: CC(=O)NCCNCc1cccc(-c2nc3c(C)c(-c4ccccc4)ccn3n2)c1]

Reference synthesis of compound 43 yields compound 50. ¹H NMR (300 MHz, Methanol-d 4) δ 7.79 (s, 1H), 7.68-7.53 (m, 3H), 7.51 (ddt, J=7.5, 2.0, 1.0 Hz, 1H), 7.44 (tt, J=1.9, 1.0 Hz, 1H), 7.41-7.31 (m, 4H), 3.83 (s, 2H), 3.32 (m, J=2.5 Hz, 2H), 2.76 (m, J=3.8 Hz, 2H), 2.36 (s, 3H), 1.90 (s, 3H).

### Example 51

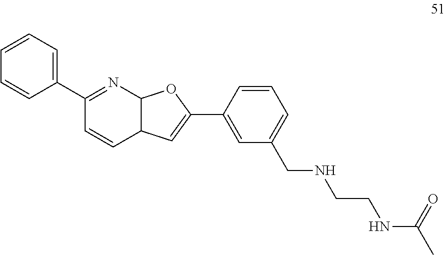 [SMILES: CC(=O)NCCNCc1cccc(C2=CC3C=CC(c4ccccc4)=NC3O2)c1]

Reference synthesis of compound 43 yields compound 51. ¹H NMR (300 MHz, Methanol-d 4) δ 7.65 (dt, J=7.5, 2.0 Hz, 1H), 7.50-7.35 (m, 5H), 7.35-7.27 (m, 2H), 7.04 (t, J=7.5 Hz, 1H), 6.33 (s, 1H), 6.10 (d, J=7.5 Hz, 2H), 3.86 (s, 2H), 3.27 (t, J=2.7 Hz, 2H), 2.73 (t, J=4.8 Hz, 2H), 1.93 (s, 3H).

### Example 52

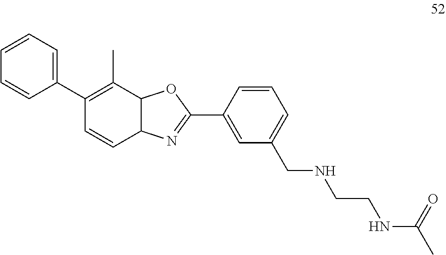 [SMILES: CC(=O)NCCNCc1cccc(C2=NC3C=CC(c4ccccc4)=C(C)C3O2)c1]

Reference to the synthesis of compound 10 yields compound 52. ¹H NMR (300 MHz, Methanol-d 4) δ 7.63 (dt, J=2.0, 1.0 Hz, 1H), 7.50-7.43 (m, 2H), 7.43-7.28 (m, 5H), 7.05 (t, J=7.5 Hz, 1H), 6.21-5.99 (m, 2H), 5.42-5.28 (m, 2H), 3.86 (s, 2H), 3.35 (t, J=3.0 Hz, 2H), 2.81 (t, J=3.9 Hz, 2H), 2.27 (s, 3H), 1.90 (s, 3H).

### Example 53

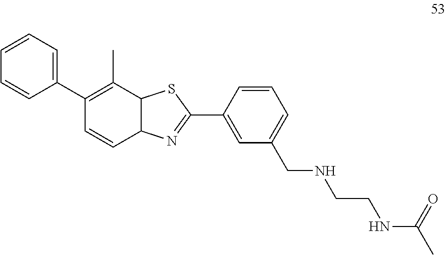 [SMILES: CC(=O)NCCNCc1cccc(C2=NC3C=CC(c4ccccc4)=C(C)C3S2)c1]

Referring to the synthesis method of the compound 10, the compound 53 can be produced. ¹H NMR (300 MHz, Methanol-d 4) δ 7.55 (ddd, J=7.5, 2.0, 0.9 Hz, 1H), 7.49-7.28 (m, 7H), 7.13 (t, J=7.5 Hz, 1H), 6.12 (p, J=1.0 Hz, 1H), 6.03 (s, 1H), 5.54 (s, 1H), 3.85 (s, 2H), 3.32 (t, J=2.4 Hz, 2H), 2.74 (t, J=3.5 Hz, 2H), 2.05 (s, 3H), 1.90 (s, 3H).

### Example 54

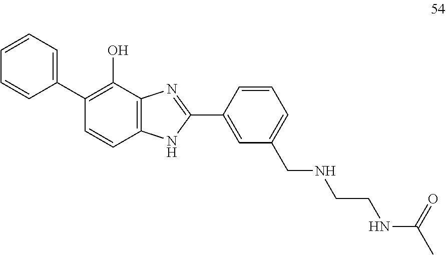 [SMILES: CC(=O)NCCNCc1cccc(-c2nc3c(O)c(-c4ccccc4)ccc3[nH]2)c1]

Referring to the synthesis method of the compound 10, the compound 54 can be produced. ¹H NMR (300 MHz, Methanol-d 4) δ 7.91 (dt, J=7.5, 2.0 Hz, 1H), 7.71 (s, 1H), 7.46 (dq, J=2.0, 1.0 Hz, 1H), 7.44-7.26 (m, 7H), 7.05 (t, J=7.5 Hz, 1H), 3.79 (s, 2H), 3.35 (t, J=2.8 Hz, 2H), 2.73 (t, J=3.5 Hz, 2H), 1.90 (s, 3H).

### Example 55

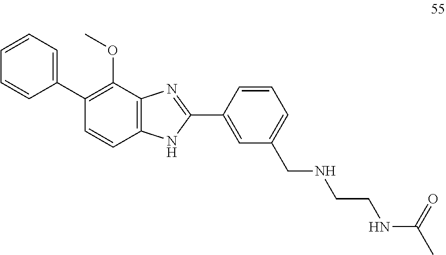 [SMILES: COc1c(-c2ccccc2)ccc2[nH]c(-c3cccc(CNCCNC(C)=O)c3)nc12]

Referring to the synthesis method of the compound 10, the compound 55 can be produced. ¹H NMR (300 MHz, Methanol-d 4) δ 7.97 (s, 1H), 7.91 (dt, J=7.5, 2.0 Hz, 1H), 7.53-7.26 (m, 8H), 7.05 (t, J=7.5 Hz, 1H), 3.85 (s, 3H), 3.79 (s, 2H), 3.35 (t, J=2.1 Hz, 2H), 2.73 (t, J=3.2 Hz, 2H), 1.90 (s, 3H).

### Example 56

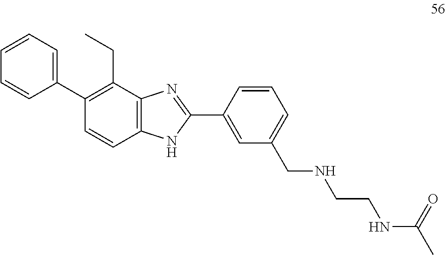 [SMILES: CCc1c(-c2ccccc2)ccc2[nH]c(-c3cccc(CNCCNC(C)=O)c3)nc12]

Referring to the synthesis method of the compound 10, the compound 56 can be produced. ¹H NMR (300 MHz, Methanol-d 4) δ 7.85 (dt, J=7.5, 2.0 Hz, 1H), 7.70 (s, 1H), 7.63 (tt, J=2.0, 1.0 Hz, 1H), 7.52 (s, 1H), 7.51-7.45 (m, 2H), 7.41 (dtt,J=7.5, 2.0, 1.0 Hz, 1H), 7.39-7.28 (m, 3H), 7.08 (t, J=7.5 Hz, 1H), 3.79 (s, 2H), 3.35 (t, J=2.4 Hz, 2H), 2.74 (dd, J=4.5 Hz, 4H), 1.90 (s, 3H), 1.09 (t, J=1.6 Hz, 3H).

### Example 57

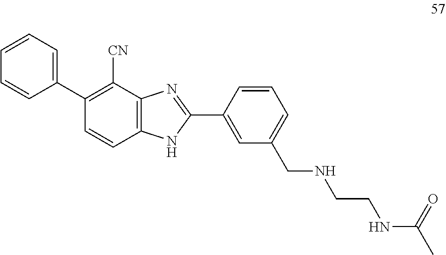 [SMILES: CC(=O)NCCNCc1cccc(-c2nc3c(C#N)c(-c4ccccc4)ccc3[nH]2)c1]

Referring to the synthesis method of the compound 10, the compound 57 can be produced. ¹H NMR (300 MHz, Methanol-d 4) δ 7.87-7.81 (m, 2H), 7.81-7.75 (m, 3H), 7.66 (tt, J=2.0, 1.1 Hz, 1H), 7.45-7.32 (m, 4H), 7.11 (t, J=7.5 Hz, 1H), 3.84 (s, 2H), 3.35 (t, J=2.0 Hz, 2H), 2.73 (t, J=2.9 Hz, 2H), 1.93 (s, 3H).

### Example 58

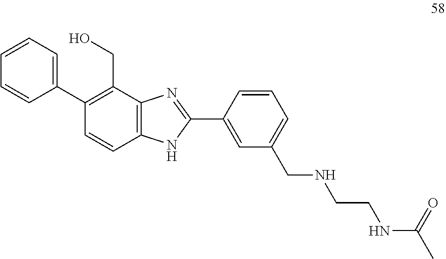 [SMILES: CC(=O)NCCNCc1cccc(-c2nc3c(CO)c(-c4ccccc4)ccc3[nH]2)c1]

Referring to the synthesis method of the compound 10, the compound 58 can be produced. ¹H NMR (300 MHz, Methanol-d 4) δ 7.85 (dt, J=7.5, 2.0 Hz, 1H), 7.75 (s, 1H), 7.64-7.56 (m, 3H), 7.47-7.38 (m, 2H), 7.37-7.28 (m, 3H), 7.12 (t, J=7.5 Hz, 1H), 4.68 (s, 2H), 3.81 (s, 2H), 3.35 (t, J=2.3 Hz, 2H), 2.73 (t, J=4.0 Hz, 2H), 1.90 (s, 3H).

### Example 59

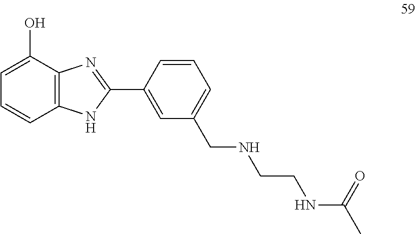 [SMILES: CC(=O)NCCNCc1cccc(-c2nc3c(O)cccc3[nH]2)c1]

Referring to the synthesis method of the compound 10, the compound 59 can be produced. ¹H NMR (300 MHz, Methanol-d 4) δ 7.44 (dq, J=2.0, 1.0 Hz, 1H), 7.41 (ddd, J=7.3, 1.9, 1.0 Hz, 1H), 7.35 (s, 1H), 7.29 (s, 1H), 7.05 (t, J=7.5 Hz, 1H), 6.72 (s, 1H), 3.76 (s, 2H), 3.34 (t, J=2.4 Hz, 2H), 2.73 (t, J=3.9 Hz, 2H), 1.93 (s, 3H).

### Example 60

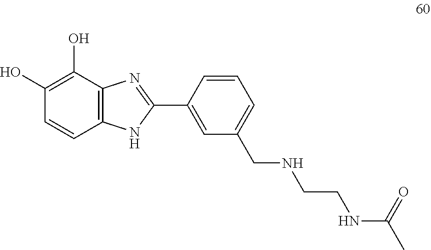 [SMILES: CC(=O)NCCNCc1cccc(-c2nc3c(O)c(O)ccc3[nH]2)c1]

Referring to the synthesis method of the compound 10, the compound 60 can be produced. ¹H NMR (300 MHz, Methanol-d 4) δ 7.85 (dt, J=7.5, 2.0 Hz, 1H), 7.44 (tt, J=2.0, 1.0 Hz, 1H), 7.41-7.34 (m, 2H), 7.07 (t, J=7.5 Hz, 1H), 6.98 (s, 1H), 3.76 (s, 2H), 3.29 (t, J=1.9 Hz, 2H), 2.73 (t, J=3.1 Hz, 2H), 1.89 (s, 3H).

### Example 61

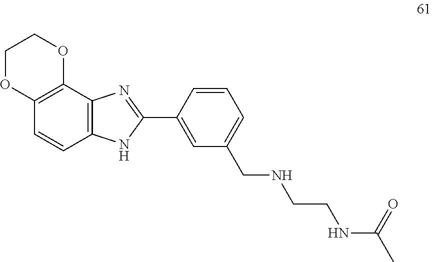 [SMILES: CC(=O)NCCNCc1cccc(-c2nc3c4c(ccc3[nH]2)OCCO4)c1]

Referring to the synthesis method of the compound 10, the compound 61 can be produced. ¹H NMR (300 MHz, Methanol-d 4) δ 7.86 (dt, J=7.3, 2.0 Hz, 1H), 7.47-7.41 (m, 2H), 7.38 (ddt, J=7.5, 2.0, 1.0 Hz, 1H), 7.05 (t, J=7.5 Hz, 1H), 6.99 (s, 1H), 4.29 (d, J=16.3 Hz, 4H), 3.79 (s, 2H), 3.29 (t, J=2.3 Hz, 2H), 2.73 (t, J=3.4 Hz, 2H), 1.93 (s, 3H).

### Example 62

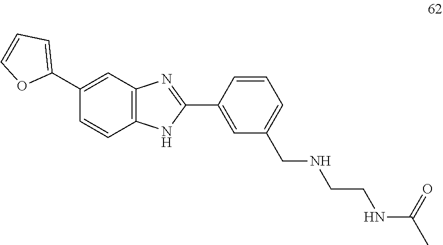 [SMILES: CC(=O)NCCNCc1cccc(-c2nc3cc(-c4ccco4)ccc3[nH]2)c1]

Referring to the synthesis method of compound 10, compound 62 can be produced. ¹H NMR (300 MHz, Methanol-d 4) δ 7.85-7.76 (m, 3H), 7.70 (d, J=26.9 Hz, 2H), 7.63 (dp, J=2.0, 1.0 Hz, 1H), 7.59 (s, 1H), 7.41 (dtt, J=7.5, 2.0, 1.0 Hz, 1H), 7.12 (t, J=7.5 Hz, 1H), 6.86 (s, 1H), 3.89 (s, 2H), 3.29 (t, J=2.9 Hz, 2H), 2.73 (t, J=3.1 Hz, 2H), 1.94 (s, 3H).

### Example 63

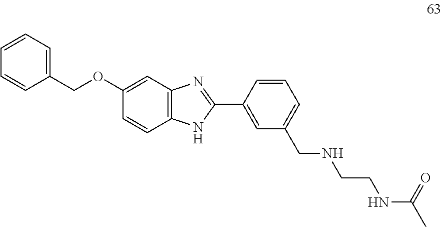 [SMILES: CC(=O)NCCNCc1cccc(-c2nc3cc(OCc4ccccc4)ccc3[nH]2)c1]

Referring to the synthesis method of the compound 10, the compound 63 can be produced. ¹H NMR (300 MHz, Methanol-d 4) δ 7.77 (dt, J=7.5, 2.0 Hz, 1H), 7.49 (s, 1H), 7.46 (tt, J=2.0, 1.0 Hz, 1H), 7.43-7.27 (m, 6H), 7.15-7.07 (m, 2H), 7.00 (s, 1H), 5.17 (d, J=1.0 Hz, 2H), 3.79 (s, 2H), 3.35 (s, 2H), 2.73 (s, 2H), 1.90 (s, 3H).

### Example 64

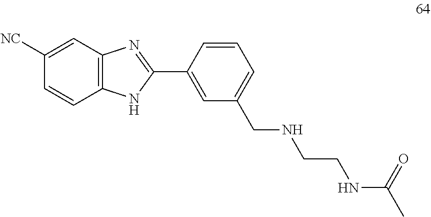 [SMILES: CC(=O)NCCNCc1cccc(-c2nc3cc(C#N)ccc3[nH]2)c1]

64, reference to the synthesis method of compound 10. ¹H NMR (500 MHz, Chloroform-d) δ 7.96 (s, 1H), 7.79 (s, 1H), 7.66-7.59 (m, 3H), 7.57 (dt, J=7.5, 2.0 Hz, 1H), 7.40 (ddt, J=7.5, 2.0, 0.9 Hz, 1H), 7.07 (t, J=7.5 Hz, 1H), 3.85-3.67 (m, 2H), 3.29 (s, 2H), 2.73 (s, 2H), 1.93 (s, 3H).

Tablet

Compound 1 (50 g), hydroxypropanmethyl cellulose E (150 g), starch (200 g), povidone K30 and magnesium stearate (1 g) in Example 1 were mixed, pelleted and pressed.

Furthermore, according to the conventional formulation method of Pharmacopoeia 2015, the compounds produced from Example 1-66 can be made into capsules, powder, granules, pills, injection, syrup, oral liquid, inhalant, ointment, ointment, suppository, or patch.

Test Case 1

Pharmacological test proved that the VISTA inhibitory activity of the invention can be used to prepare antitumor drugs. The following are the pharmacological experimental results of some compounds of the present invention:
1. Ability ability of compounds to VISTA protein
(I) Experimental Equipment and Reagents
1. The machine model used in this experiment is: Biacore S200.
2. S-series CM5 chip. Goods number: 29-1049-88 (one piece), BR-1005-30 (three pieces), 29-1496-03 (ten pieces), the purchase place is GE Healthcare.
3, amino coupling kit. Goods number: BR-1000-50, the purchase place is GE Healthcare.
4. Buffer: 10×PBS-P+(cargo number: 28-9950-84), and the purchase place is GE Healthcare.
5, analyze pure DMSO, deionized water (0.22 μm membrane filtration).
6, Protein: VISTA protein modified by glycosylation.
7. Other consumables: uncovered 1.5 ml EP tube (Goods No.: BR-1002-87), rubber bottle cap type 2 (cargo No.: BR-1004-11), 96-well plate (cargo No.: BR-1005-03), 96-well plate sealing film (Goods No.: 28-9758-16), the purchase place is GE Healthcare.(two) experimental procedure

To test the binding ability of compound to VISTA protein using Biacore S200 system and CM5 chip, 10 mM compound was diluted in 1.05*PBS-P to 5 concentration gradient (5 μ M, 2.5 μ M, 1.25 μ M, 0.625 μ M), and fitted to obtain compound K_{D} numeric value.
(3) K of some compounds_{D} The values are as follows:

| chemical | | chemical | |
|---|---|---|---|
| compound | K_{D}(nM) | compound | K_{D}(nM) |
| 1 | 153 | 3 | 215 |
| 7 | 461 | 10 | 338 |

2. Determination of the inhibition effect of the compounds against VISTA interaction:
(1) Experimental Equipment and Reagents
1. ELISA, the kit was purchased at R & D Systems (CAT #DY-285) to detect the release of IFN-γ, anti-human CD3 antibody, recombinant human VISTA protein (R & D Systems, CAT #7126-B7)
2. SpectraMax i3X Multifunction microplate reader (Molecular Device)
3. 384 shallow hole plate (Nunc, CAT #264706)
(2) Experimental Method
1. Experimental procedure:
1.1 Recombinant human VISTA (2.5 μg/ml) and anti-human CD3 antibody (2.5 μg/ml) were added to 96-well plates and stored in 4 C overnight.
1.2 The next day, the anti-human VISTA antibody was added to the co-incubation for 30 min. Thereafter, cells were washed with 1*PBS and test compounds were added for 30 min. Isolated PBMC (0.1*106 cell/well) and anti-human CD28 antibody (1 μg/ml) were added to the test wells. At 37 C, 5% CO₂Below the conditions, the incubation was continued for 72 h.
1.3 After separation by centrifugation at 4 C, 200 g*5 min, the supernatant was collected. The IFN-γ release was then determined by using the IFN-γ assay with ELISA.
1.4, the inhibition rate of compound VISTA was the rescue rate of IFN-γ release in human PBMC cells.
(3) Experimental Results

The following table shows the activity range or IC of the compounds against VISTA interaction inhibitory activity₅₀. The ranges is as follows: A=1 nM-100 nM; B=100.01 nM-1000 nM; C=1001-10000 nM.

| chemical | |
|---|---|
| compound | IC₅₀(nM) |
| 1 | A |
| 2 | B |
| 3 | A |
| 4 | A |
| 5 | A |
| 6 | A |
| 7 | A |
| 8 | B |
| 9 | A |
| 10 | B |
| 11 | A |
| 12 | B |
| 13 | A |
| 14 | B |
| 15 | A |
| 16 | A |
| 17 | A |
| 18 | A |
| 19 | B |
| 20 | A |
| 21 | B |
| 22 | A |
| 23 | A |
| 24 | A |
| 25 | B |
| 26 | B |
| 27 | A |
| 28 | A |
| 29 | A |
| 30 | A |
| 31 | A |
| 32 | A |
| 33 | A |
| 34 | A |
| 35 | B |
| 36 | A |
| 37 | B |
| 38 | A |
| 39 | B |
| 40 | B |
| 41 | B |
| 42 | B |
| 43 | B |
| 44 | B |
| 45 | A |
| 46 | A |
| 47 | A |
| 48 | B |
| 49 | B |
| 50 | B |
| 51 | B |
| 52 | B |
| 53 | B |
| 54 | B |
| 55 | A |
| 56 | A |
| 57 | A |
| 58 | B |
| 59 | A |
| 60 | A |
| 61 | B |
| 62 | B |
| 63 | B |
| 64 | A |

1. A heterocyclic compound shown in formula I, a pharmaceutically acceptable salt, racemic, photoisomers or solvent compound: 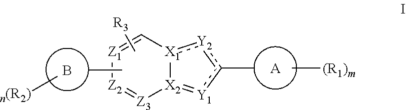
loops A and B are independently aromatic or heteroaromatic;
X₁, X₂, Z₁, Z₂, Z₃ independently for either C or N, Y₁, Y₂ independently for C, N, S or O;
each R₁ independently is selected from hydrogen, deuterium, substituted or unsubstituted hydroxyl, substituted or unsubstituted amino, halogen, substituted or unsubstituted alkyl or substituted or unsubstituted alkoxy, amino acids;
each R₂ independent is selected from hydrogen, deuterium or unsubstituted hydroxyl, substituted or unsubstituted amino, halogen, substituted or unsubstituted alkyl, substituted or unsubstituted alkoxy, or two adjacent R₂ forming substituted or unsubstituted C₄₋₇ carbon ring or heterocycles with two atoms of the B ring;
R₃ independent is selected from hydrogen, deuterium, cyanide, halogen, vinyl, trifluoromethyl, methoxy, or C₁₋₄ alkyl;
m is 1, 2, or 3;
n is 1 or 2.

2. The heterocyclic compound according to claim 1, wherein each R₁, the substituted alkyl or substituted alkoxy group can be one or more of the following groups: halogen, C₁₋₄ alkyl, hydroxyl groups, and C₁₋₄ alkoxy, cyanide, trifluoromethyl, C₁₋₄ carboxyl, C₁₋₄ ester group or C₁₋₄ amide group; the substituent in the substituted hydroxyl or substituted amino group can be one or more of the following groups: C₁₋₈ alkyl, C₁₋₈ acylamino, C₁₋₈ ester group, C₁₋₈ carboxyl, C₁₋₈ hydroxyl group; wherein the C₁₋₈ alkyl, C₁₋₈ acylamino, C₁₋₈ ester group, C₁₋₈ carboxyl, C₁₋₈ hydroxyl group may optionally be substituted by one or more of the following substituents: hydroxyl, carboxyl, cyanide, amino, cyclic alkyl, aryl, heterocyclic, alkenyl, alkyne group; when the substituents are multiple, the substituents are the same or different.

3. The heterocyclic compound according to claim 1, wherein each R₂, the substituted alkyl or substituted alkoxy group can be one or more of the following groups: halogen, C₁₋₄ alkyl, hydroxyl groups, and C₁₋₄ alkoxy, cyanide, trifluoromethyl, C₁₋₄ carboxyl, C₁₋₄ ester group or C₁₋₄ amide group; the substituent in the substituted hydroxyl or substituted amino group is one or more of the following groups: C₁₋₈ alkyl, C₁₋₈ acylamino, C₁₋₈ ester group, C₁₋₈ carboxyl, C₁₋₈ hydroxyl group; wherein the C₁₋₈ alkyl, C₁₋₈ acylamino, C₁₋₈ ester group, C₁₋₈ carboxyl, C₁₋₈ hydroxyl group may optionally be substituted by one or more of the following substituents: hydroxyl, carboxyl, cyanide, amino, cycloalkyl, aryl, heterocyclic, alkenyl, alkyne; when the two adjacent R₂ and the two atoms in the B ring form a 4-7 substituted carbon ring or substituted heterocyclic, the substituted carbon ring or substituted heterocyclic substituent is one or more of the following groups: Halogens, C₁₋₄ alkyl, hydroxyl groups, and C₁₋₄ alkoxy, cyanide, trifluoromethyl, C₁₋₄ carboxyl, C₁₋₄ ester group or C₁₋₄ amide group; when the substituents are multiple, the substituent is the same or different.

4. The heterocyclic compound according to claim 1, wherein the compound is selected from the following compounds 1-64:

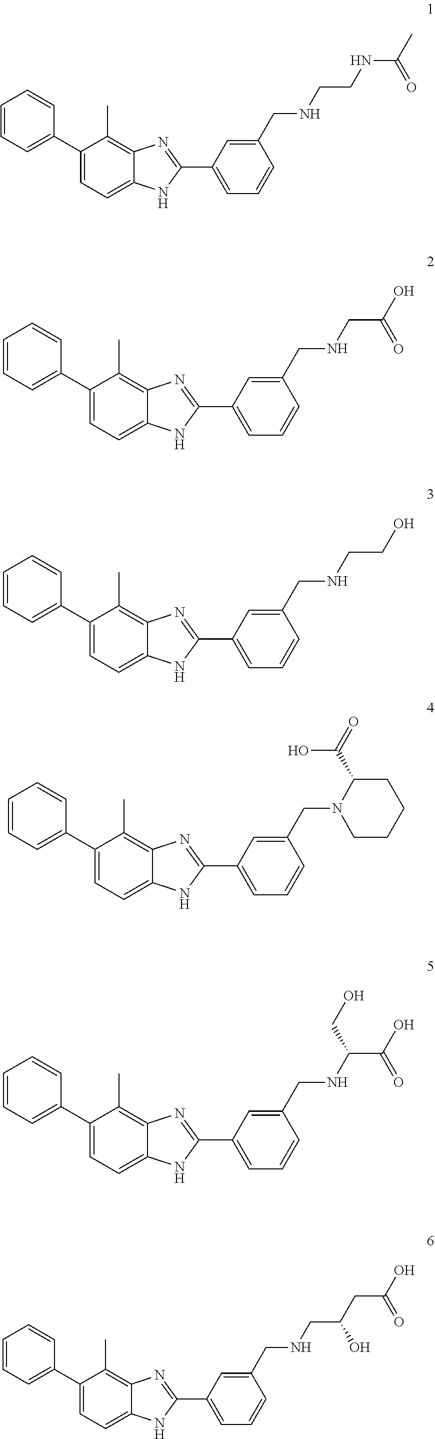 [SMILES: CC(=O)NCCNCc1cccc(-c2nc3c(C)c(-c4ccccc4)ccc3[nH]2)c1 ; Cc1c(-c2ccccc2)ccc2[nH]c(-c3cccc(CNCC(=O)O)c3)nc12 ; Cc1c(-c2ccccc2)ccc2[nH]c(-c3cccc(CNCCO)c3)nc12 ; Cc1c(-c2ccccc2)ccc2[nH]c(-c3cccc(CN4CCCC[C@H]4C(=O)O)c3)nc12 ; Cc1c(-c2ccccc2)ccc2[nH]c(-c3cccc(CN[C@H](CO)C(=O)O)c3)nc12 ; Cc1c(-c2ccccc2)ccc2[nH]c(-c3cccc(CNC[C@@H](O)CC(=O)O)c3)nc12]

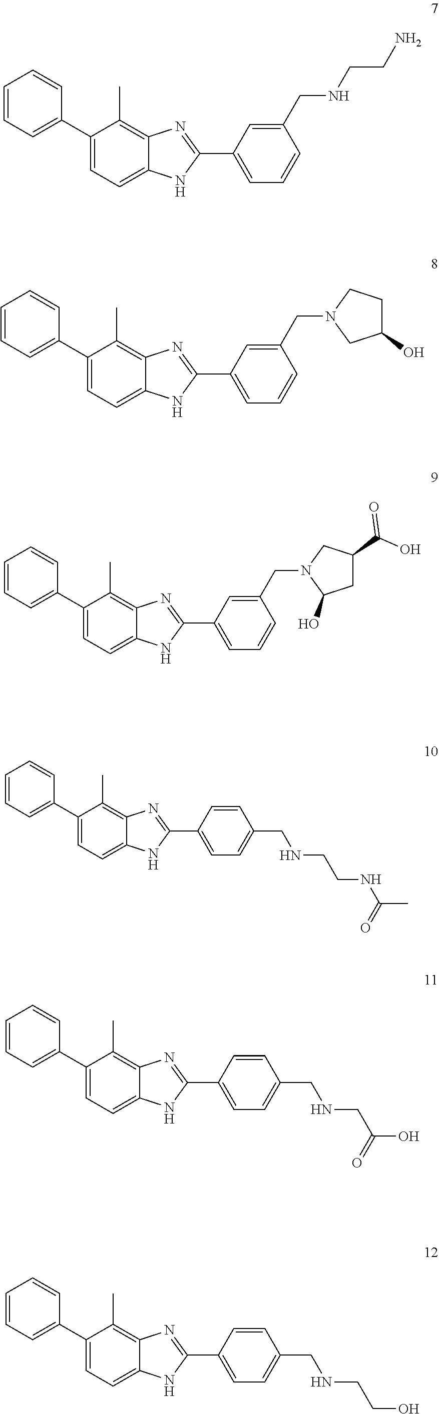 [SMILES: Cc1c(-c2ccccc2)ccc2[nH]c(-c3cccc(CNCCN)c3)nc12 ; Cc1c(-c2ccccc2)ccc2[nH]c(-c3cccc(CN4CC[C@@H](O)C4)c3)nc12 ; Cc1c(-c2ccccc2)ccc2[nH]c(-c3cccc(CN4C[C@@H](C(=O)O)C[C@H]4O)c3)nc12 ; CC(=O)NCCNCc1ccc(-c2nc3c(C)c(-c4ccccc4)ccc3[nH]2)cc1 ; Cc1c(-c2ccccc2)ccc2[nH]c(-c3ccc(CNCC(=O)O)cc3)nc12 ; Cc1c(-c2ccccc2)ccc2[nH]c(-c3ccc(CNCCO)cc3)nc12]

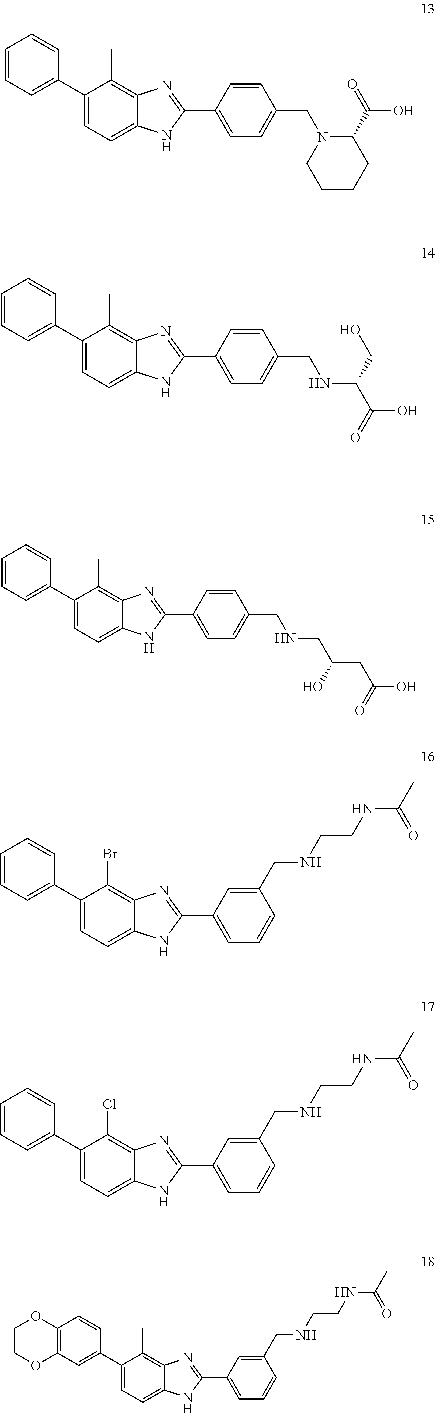 [SMILES: Cc1c(-c2ccccc2)ccc2[nH]c(-c3ccc(CN4CCCC[C@H]4C(=O)O)cc3)nc12 ; Cc1c(-c2ccccc2)ccc2[nH]c(-c3ccc(CN[C@H](CO)C(=O)O)cc3)nc12 ; Cc1c(-c2ccccc2)ccc2[nH]c(-c3ccc(CNC[C@@H](O)CC(=O)O)cc3)nc12 ; CC(=O)NCCNCc1cccc(-c2nc3c(Br)c(-c4ccccc4)ccc3[nH]2)c1 ; CC(=O)NCCNCc1cccc(-c2nc3c(Cl)c(-c4ccccc4)ccc3[nH]2)c1 ; CC(=O)NCCNCc1cccc(-c2nc3c(C)c(-c4ccc5c(c4)OCCO5)ccc3[nH]2)c1]

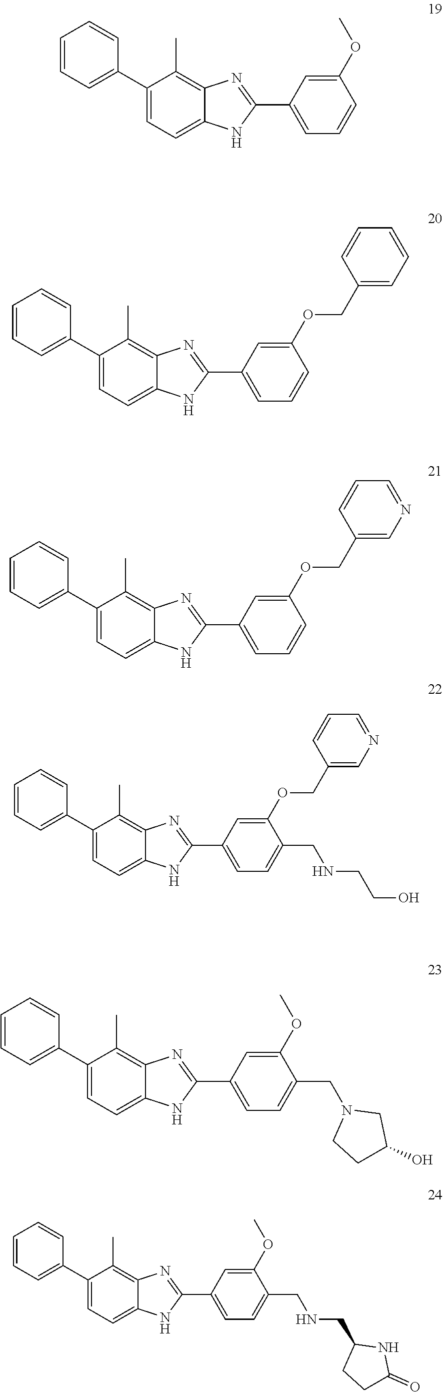 [SMILES: COc1cccc(-c2nc3c(C)c(-c4ccccc4)ccc3[nH]2)c1 ; Cc1c(-c2ccccc2)ccc2[nH]c(-c3cccc(OCc4ccccc4)c3)nc12 ; Cc1c(-c2ccccc2)ccc2[nH]c(-c3cccc(OCc4cccnc4)c3)nc12 ; Cc1c(-c2ccccc2)ccc2[nH]c(-c3ccc(CNCCO)c(OCc4cccnc4)c3)nc12 ; COc1cc(-c2nc3c(C)c(-c4ccccc4)ccc3[nH]2)ccc1CN1CC[C@@H](O)C1 ; COc1cc(-c2nc3c(C)c(-c4ccccc4)ccc3[nH]2)ccc1CNC[C@@H]1CCC(=O)N1]

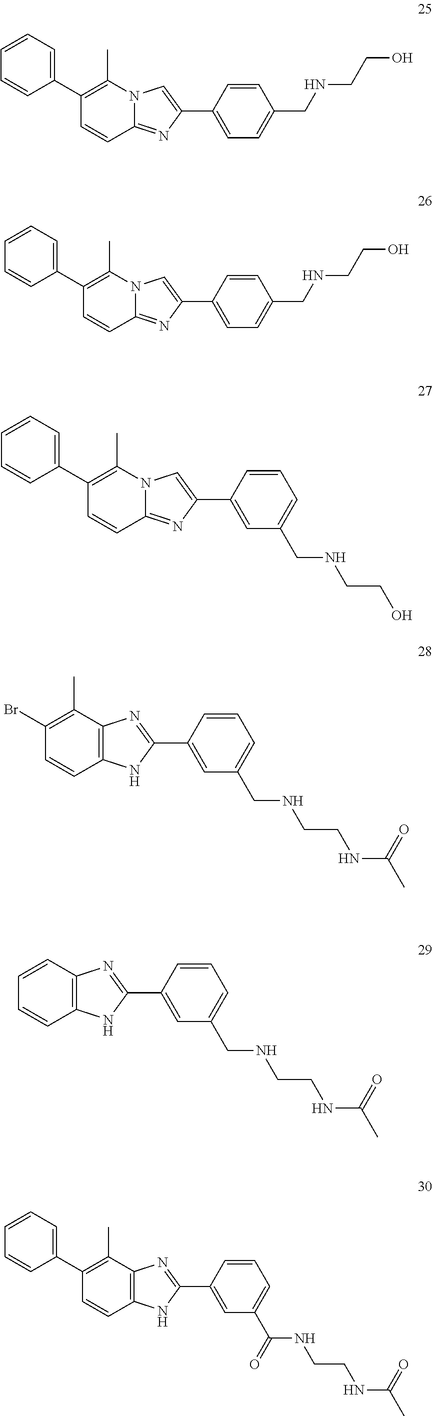 [SMILES: Cc1c(-c2ccccc2)ccc2nc(-c3ccc(CNCCO)cc3)cn12 ; Cc1c(-c2ccccc2)ccc2nc(-c3ccc(CNCCO)cc3)cn12 ; Cc1c(-c2ccccc2)ccc2nc(-c3cccc(CNCCO)c3)cn12 ; CC(=O)NCCNCc1cccc(-c2nc3c(C)c(Br)ccc3[nH]2)c1 ; CC(=O)NCCNCc1cccc(-c2nc3ccccc3[nH]2)c1 ; CC(=O)NCCNC(=O)c1cccc(-c2nc3c(C)c(-c4ccccc4)ccc3[nH]2)c1]

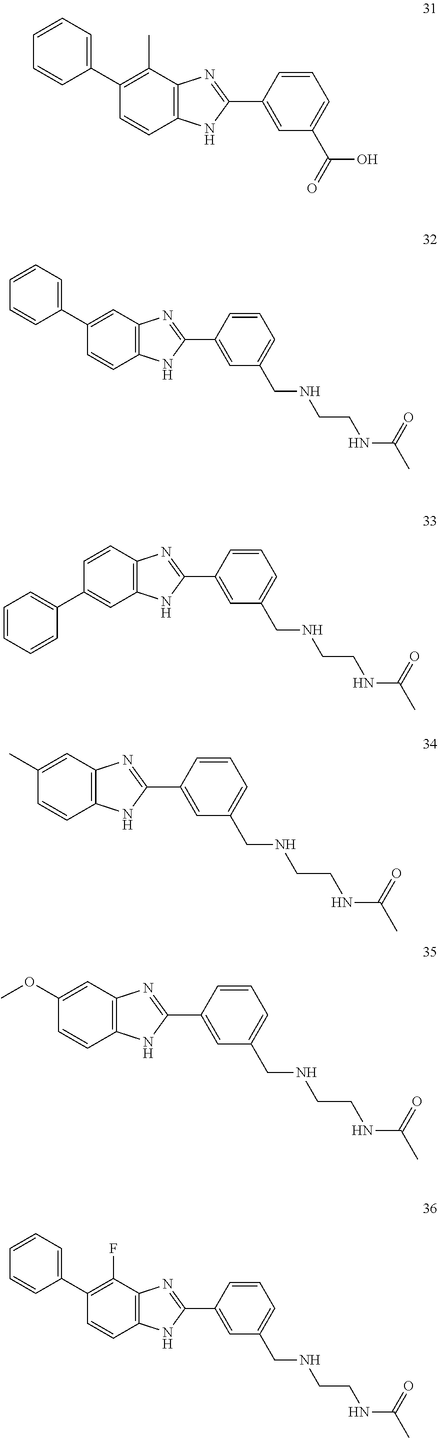 [SMILES: Cc1c(-c2ccccc2)ccc2[nH]c(-c3cccc(C(=O)O)c3)nc12 ; CC(=O)NCCNCc1cccc(-c2nc3cc(-c4ccccc4)ccc3[nH]2)c1 ; CC(=O)NCCNCc1cccc(-c2nc3ccc(-c4ccccc4)cc3[nH]2)c1 ; CC(=O)NCCNCc1cccc(-c2nc3cc(C)ccc3[nH]2)c1 ; COc1ccc2[nH]c(-c3cccc(CNCCNC(C)=O)c3)nc2c1 ; CC(=O)NCCNCc1cccc(-c2nc3c(F)c(-c4ccccc4)ccc3[nH]2)c1]

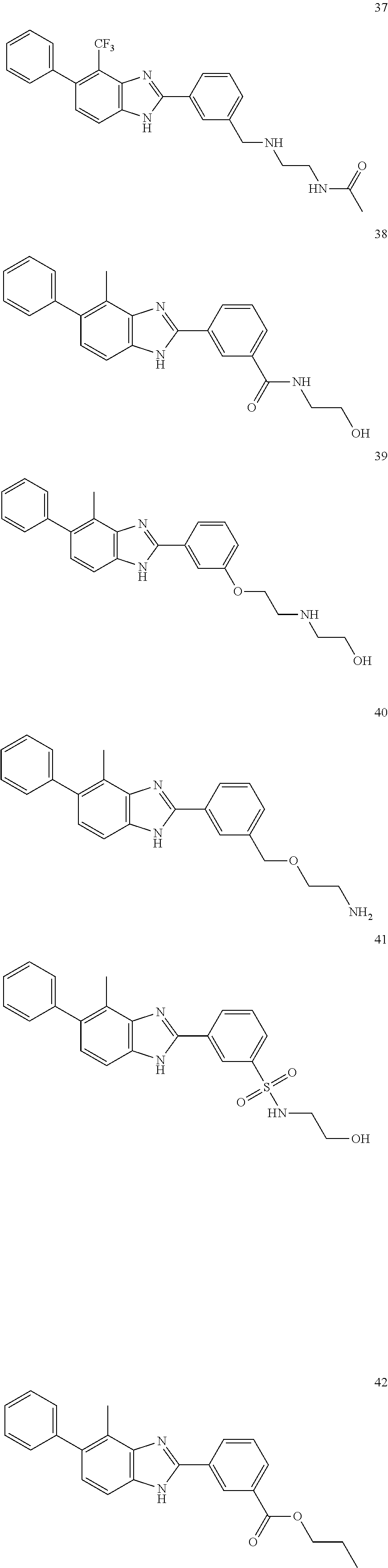 [SMILES: CC(=O)NCCNCc1cccc(-c2nc3c(C(F)(F)F)c(-c4ccccc4)ccc3[nH]2)c1 ; Cc1c(-c2ccccc2)ccc2[nH]c(-c3cccc(C(=O)NCCO)c3)nc12 ; Cc1c(-c2ccccc2)ccc2[nH]c(-c3cccc(OCCNCCO)c3)nc12 ; Cc1c(-c2ccccc2)ccc2[nH]c(-c3cccc(COCCN)c3)nc12 ; Cc1c(-c2ccccc2)ccc2[nH]c(-c3cccc(S(=O)(=O)NCCO)c3)nc12 ; CCCOC(=O)c1cccc(-c2nc3c(C)c(-c4ccccc4)ccc3[nH]2)c1]

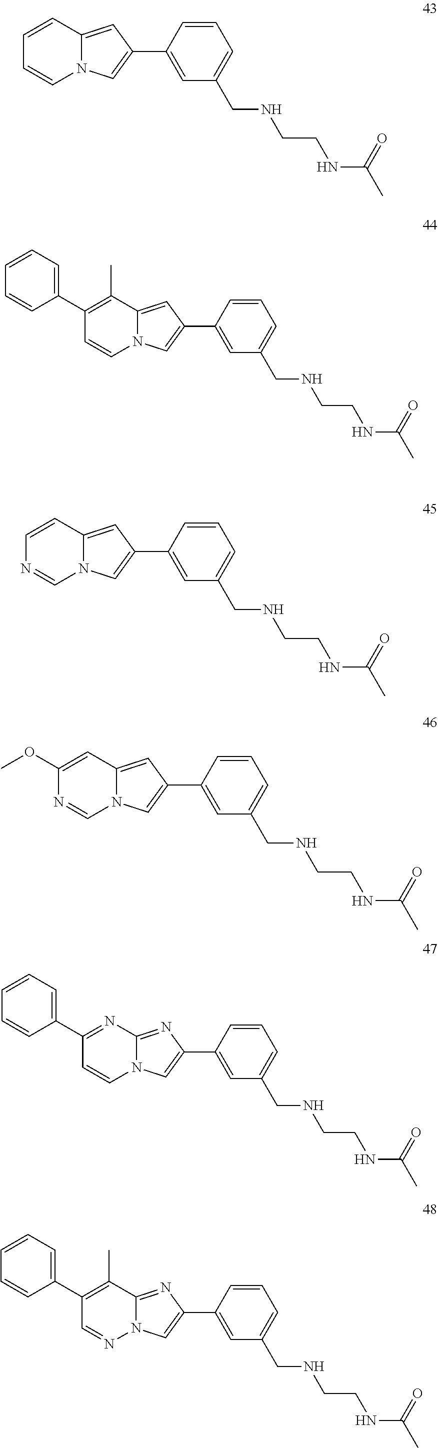 [SMILES: CC(=O)NCCNCc1cccc(-c2cc3ccccn3c2)c1 ; CC(=O)NCCNCc1cccc(-c2cc3c(C)c(-c4ccccc4)ccn3c2)c1 ; CC(=O)NCCNCc1cccc(-c2cc3ccncn3c2)c1 ; COc1cc2cc(-c3cccc(CNCCNC(C)=O)c3)cn2cn1 ; CC(=O)NCCNCc1cccc(-c2cn3ccc(-c4ccccc4)nc3n2)c1 ; CC(=O)NCCNCc1cccc(-c2cn3ncc(-c4ccccc4)c(C)c3n2)c1]

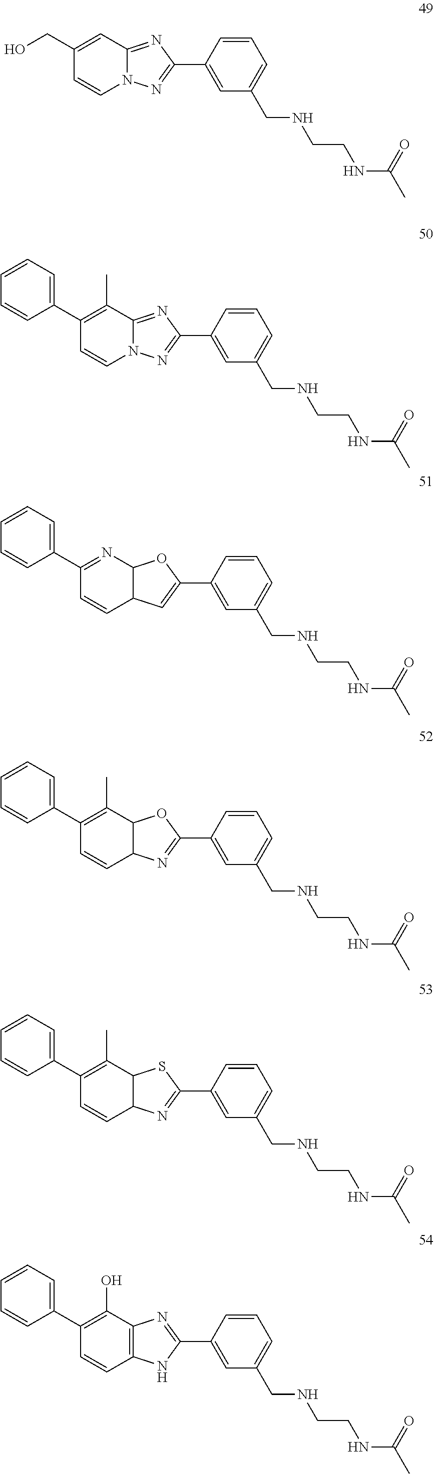 [SMILES: CC(=O)NCCNCc1cccc(-c2nc3cc(CO)ccn3n2)c1 ; CC(=O)NCCNCc1cccc(-c2nc3c(C)c(-c4ccccc4)ccn3n2)c1 ; CC(=O)NCCNCc1cccc(C2=CC3C=CC(c4ccccc4)=NC3O2)c1 ; CC(=O)NCCNCc1cccc(C2=NC3C=CC(c4ccccc4)=C(C)C3O2)c1 ; CC(=O)NCCNCc1cccc(C2=NC3C=CC(c4ccccc4)=C(C)C3S2)c1 ; CC(=O)NCCNCc1cccc(-c2nc3c(O)c(-c4ccccc4)ccc3[nH]2)c1]

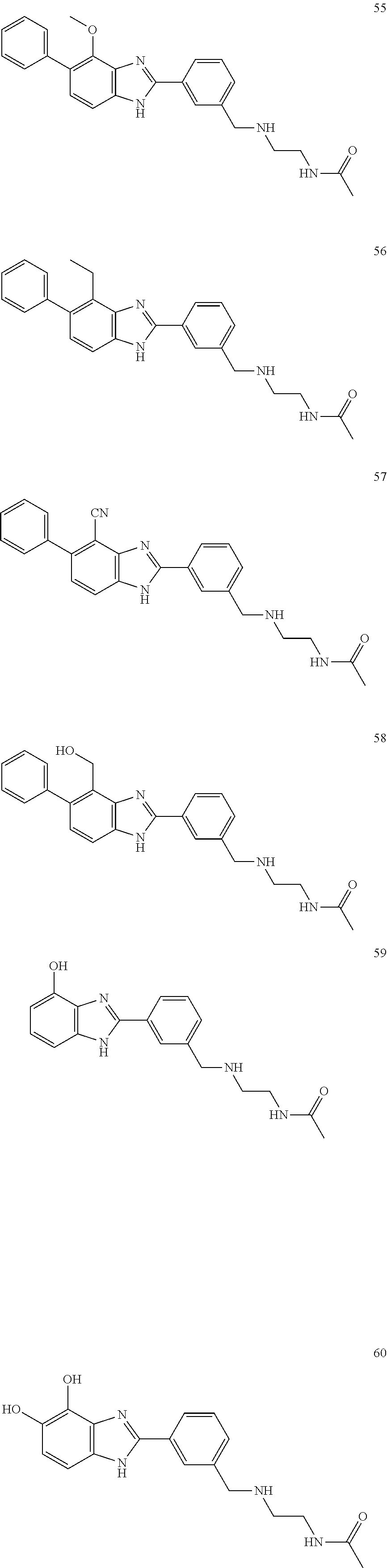 [SMILES: COc1c(-c2ccccc2)ccc2[nH]c(-c3cccc(CNCCNC(C)=O)c3)nc12 ; CCc1c(-c2ccccc2)ccc2[nH]c(-c3cccc(CNCCNC(C)=O)c3)nc12 ; CC(=O)NCCNCc1cccc(-c2nc3c(C#N)c(-c4ccccc4)ccc3[nH]2)c1 ; CC(=O)NCCNCc1cccc(-c2nc3c(CO)c(-c4ccccc4)ccc3[nH]2)c1 ; CC(=O)NCCNCc1cccc(-c2nc3c(O)cccc3[nH]2)c1 ; CC(=O)NCCNCc1cccc(-c2nc3c(O)c(O)ccc3[nH]2)c1]

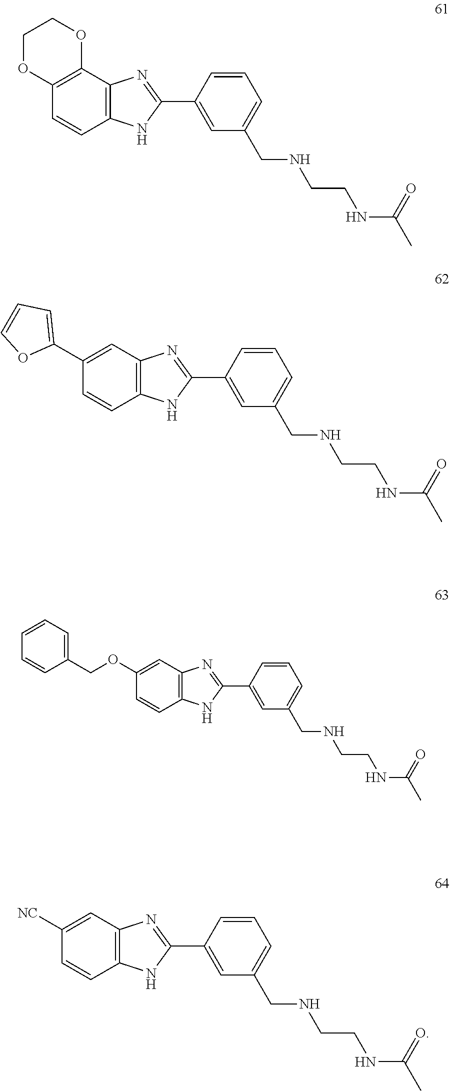 [SMILES: CC(=O)NCCNCc1cccc(-c2nc3c4c(ccc3[nH]2)OCCO4)c1 ; CC(=O)NCCNCc1cccc(-c2nc3cc(-c4ccco4)ccc3[nH]2)c1 ; CC(=O)NCCNCc1cccc(-c2nc3cc(OCc4ccccc4)ccc3[nH]2)c1 ; CC(=O)NCCNCc1cccc(-c2nc3cc(C#N)ccc3[nH]2)c1]

5. The heterocyclic compound according to claim 1, characterized in, when X₁, X₂, Z₁, Z₂, Z₃ is C, Y₁ and Y₂ is N, the synthesis route of the described compound is shown as follows: 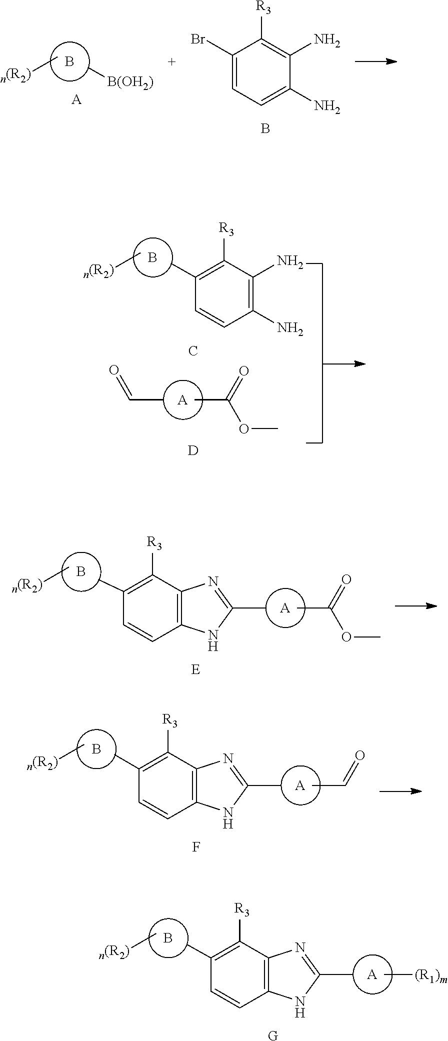
the synthesis steps are as follows:
(1) compound A and B were coupled under Suzuki coupling condition to obtain compound C;
(2) compounds D reacted with C to obtain compound E via condensation;
(3) compound E was reduced to obtain compound F;
(4) compound F was accomplished under reductive amination reaction to obtain compound G.

6-8. (canceled)

9. A pharmaceutical composition containing a heterocyclic compound of claim 1 or a pharmaceutically acceptable salt, racemic, optical isomer or solvent compound thereof as an active ingredient and a pharmaceutically acceptable carrier.

10. The pharmaceutical composition according to claim 9, wherein the drug composition is a capsule, powder, tablet, granule, bolus, injection, syrup, oral solution, inhalant, inhalant, ointment, suppository or patch.

11. A method for treating an immune-related disease in a subject in need thereof, the method comprising administering to the subject an effective amount of the heterocyclic compound according to claim 1 or pharmaceutically acceptable salts, racemic, photoisomers or solvent compounds thereof.

12. The method of claim 11, wherein the immune-related disease is selected from tumor.

13. A method for treating an immune-related disease in a subject in need thereof, the method comprising administering to the subject an effective amount of the heterocyclic compound according to claim 3 or pharmaceutically acceptable salts, racemic, photoisomers or solvent compounds thereof.

14. The method of claim 8, wherein the immune-related disease is selected from tumor.

15. A pharmaceutical composition containing a heterocyclic compound of claim 4 or a pharmaceutically acceptable salt, racemic, optical isomer or solvent compound thereof as an active ingredient and a pharmaceutically acceptable carrier.

16. The pharmaceutical composition according to claim 15, wherein the drug composition is a capsule, powder, tablet, granule, bolus, injection, syrup, oral solution, inhalant, inhalant, ointment, suppository or patch.
